(19) United States
(12) Patent Application Publication
(10) Pub. No.: US 2025/0378934 A1
(43) Pub. Date: Dec. 11, 2025

(54) Generative Model Based Health and Activity Recommendations

(71) Applicant: Google LLC, Mountain View, CA (US)

(72) Inventors: Naghmeh Rezaei, Redwood City, CA (US); Geza Kovacs, Mountain View, CA (US); Hao-Wei Su, Cambridge, MA (US); Jiening Zhan, Sunnyvale, CA (US)

(21) Appl. No.: 19/233,986

(22) Filed: Jun. 10, 2025

Related U.S. Application Data
(60) Provisional application No. 63/658,244, filed on Jun. 10, 2024.

(51) Int. Cl. G16H 20/60; G06N 20/00; G16H 10/40; G16H 15/00

(52) U.S. Cl. CPC G16H 20/60; G06N 20/00; G16H 10/40; G16H 15/00

(57) ABSTRACT

Methods, systems, devices, and non-transitory computer readable media for processing health data are provided. The disclosed technology can include receiving queries comprising health information. Based on inputting the queries into one or more machine-learned models, topics of the queries, key metrics of the health data, and analytical techniques based on the topics and the key metrics can be determined. Based on performing the analytical techniques on at least the health data comprising the key metrics, analytical results can be determined. Based on inputting the analytical results into the one or more machine-learned models, an analysis comprising explanations of the analytical results can be generated. Furthermore, visualizations based on the analysis can be generated.

### PRIORITY CLAIM

The present application is based on and claims priority to U.S. Provisional Application No. 63/658,244 which has a filing date of Jun. 10, 2024. The present application claims priority to and the benefit of such application and incorporates such application herein by reference in its entirety.

### FIELD

The present disclosure relates generally to processing health data. More particularly, the present disclosure relates to the use of generative models to parse natural language queries and generate an explanatory analysis of health data and visualizations that support the analysis.

### BACKGROUND

Various types of computing devices can be used to monitor and detect the physical states of a user. The computing devices can then analyze values associated with the physical states that were monitored and detected. Based on these values, a variety of different types of information can be used to determine the activities performed by a user. Further, the user can review this information and focus on certain information that the user may deem to be significant. However, certain users may find that locating specific information is difficult or time consuming. As such, there can be different approaches that are used to review information that is related to the activities of a user.

### SUMMARY

Aspects and advantages of embodiments of the present disclosure will be set forth in part in the following description, or can be learned from the description, or can be learned through practice of the embodiments.

One example aspect of the present disclosure is directed to a computer-implemented method of processing health data. The computer-implemented method can comprise receiving, by a computing system comprising one or more processors, one or more queries associated with health data comprising health information. The computer-implemented method can comprise determining, by the computing system, based on inputting the one or more queries into one or more machine-learned models, one or more topics of the one or more queries, one or more key metrics of the health data, and one or more analytical techniques based on the one or more topics and the one or more key metrics. The computer-implemented method can comprise determining, by the computing system, based on performing the one or more analytical techniques on at least the health data comprising the one or more key metrics, one or more analytical results. The computer-implemented method can comprise generating, by the computing system, based on inputting the one or more analytical results into the one or more machine-learned models, an analysis comprising one or more explanations of the one or more analytical results. The computer-implemented method can comprise generating, by the computing system, one or more visualizations based on the analysis.

Another example aspect of the present disclosure is directed to one or more tangible non-transitory computer-readable media storing computer-readable instructions that when executed by one or more processors cause the one or more processors to perform operations. The operations can comprise receiving one or more queries associated with health data comprising health information. The operations can comprise determining, based on inputting the one or more queries into one or more machine-learned models, one or more topics of the one or more queries, one or more key metrics of the health data, and one or more analytical techniques based on the one or more topics and the one or more key metrics. The operations can comprise determining, based on performing the one or more analytical techniques on at least the health data comprising the one or more key metrics, one or more analytical results. The operations can comprise generating, based on inputting the one or more analytical results into the one or more machine-learned models, an analysis comprising one or more explanations of the one or more analytical results. The operations can comprise generating one or more visualizations based on the analysis.

Another example aspect of the present disclosure is directed to a computing system comprising: one or more processors; one or more non-transitory computer-readable media storing instructions that when executed by the one or more processors cause the one or more processors to perform operations. The operations can comprise receiving one or more queries associated with health data comprising health information. The operations can comprise determining, based on inputting the one or more queries into one or more machine-learned models, one or more topics of the one or more queries, one or more key metrics of the health data, and one or more analytical techniques based on the one or more topics and the one or more key metrics. The operations can comprise determining, based on performing the one or more analytical techniques on at least the health data comprising the one or more key metrics, one or more analytical results. The operations can comprise generating, based on inputting the one or more analytical results into the one or more machine-learned models, an analysis comprising one or more explanations of the one or more analytical results. The operations can comprise generating one or more visualizations based on the analysis.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1A depicts a block diagram of an example computing system that generates health analyses and visualizations according to example embodiments of the present disclosure.

FIG. 1B depicts a block diagram of an example computing device that generates health analyses and visualizations according to example embodiments of the present disclosure.

FIG. 1C depicts a block diagram of an example computing device that generates health analyses and visualizations according to example embodiments of the present disclosure.

FIG. 2 depicts a block diagram of examples of machine-learned models according to example embodiments of the present disclosure.

FIG. 3 depicts an example of a computing device according to example embodiments of the present disclosure.

FIG. 4 depicts a block diagram of examples of operations performed by machine-learned models according to example embodiments of the present disclosure.

FIG. 5 depicts an example of a computing device and user interface for generating health analyses and visualizations according to example embodiments of the present disclosure.

FIG. 6 depicts an example of a computing device and user interface for generating health analyses and visualizations according to example embodiments of the present disclosure.

FIG. 7 depicts an example of a computing device and user interface for generating health analyses and visualizations according to example embodiments of the present disclosure.

FIG. 8 depicts an example of a computing device and user interface for generating health analyses and visualizations according to example embodiments of the present disclosure.

FIG. 9 depicts an example of a computing device and user interface for generating health analyses and visualizations according to example embodiments of the present disclosure.

FIG. 10 depicts an example of a computing device and user interface for generating health analyses and visualizations according to example embodiments of the present disclosure.

FIG. 11 depicts a flow chart diagram of an example method to generate health analyses and visualizations according to example embodiments of the present disclosure.

FIG. 12 depicts a flow chart diagram of an example method to generate health analyses and visualizations according to example embodiments of the present disclosure.

Reference numerals that are repeated across plural figures are intended to identify the same features in various implementations.

### DETAILED DESCRIPTION

In general, the present disclosure is directed to generating a personalized analysis of data including data relating to the health, activities, and nutrition of a user (e.g., the health data of the user of a wearable computing device) based on queries received from the user. In particular, the disclosed technology can generate an analysis (e.g., a statistical analysis) of health data that identifies and explains the significance of key health metrics of the health data based on topics and analytical techniques determined from the query. Further, the disclosed technology can implement machine-learned models (e.g., large language models (LLMs)) that have been configured and/or trained to parse the queries of a user and generate natural language explanations of an analysis of the user's health data as well as visualizations (e.g., charts and/or infographics) that can improve a user's understanding of the health data.

For example, a user can send a query associated with the user's health and activities to a computing system (e.g., a health data computing system) that is configured to receive and process such queries. The query can, for example, include questions from the user regarding relationships between a user's health metrics, physical activities, and/or nutritional habits. The computing system that processes the query can implement one or more machine-learned models (e.g., generative models including LLMs) that are configured and/or trained to parse the query and determine various information that can be used to generate an analysis of the user's health data.

For example, based on inputting the query and/or health data (e.g., health data of the user making the query) into the one or more machine-learned models, the one or more machine-learned models can determine topics associated with the query (e.g., the subject of the query, a range of dates indicated in the query, and/or the type of information that is being indicated in the query). Further, the one or more machine-learned models can determine key metrics associated with the health data. For example, the key metrics can include metrics in the health data that are determined based on the query (e.g., metrics that are directly mentioned in the query, metrics that are indirectly mentioned in the query, and/or metrics that are associated with metrics that are mentioned in the query). For example, if a query asks for information about a user's sleep patterns, the key metrics can include metrics associated with a user's nightly sleep duration and/or bedtimes of the user as well as metrics that may influence sleep patterns such as nutritional metrics (e.g., mealtimes) and/or activity metrics (e.g., the times as which activities are performed).

The one or more machine-learned models can also determine one or more analytical techniques to use on the one or more key metrics. The one or more analytical techniques can be based on the one or more topics and/or key metrics and can include statistical analysis techniques that are used to generate statistical results that can address the user's query. For example, the one or more analytical techniques can include a linear regression analysis to estimate the relationship between key metrics including sleep times and mealtimes.

The computing system can then determine analytical results based on performing the analytical techniques on health data comprising the key metrics. For example, the computing system can determine that there is a close relationship between a user's bedtimes and the user's mealtimes. By way of further example, the computing system can determine that cating dinner at a later hour may be correlated with a later bedtime.

The disclosed technology can generate an analysis that includes explanations of the analytical results. For example, the analysis can include a natural language explanation of how a user's nutritional habits may be influencing the user's sleep patterns or an explanation of how a user's heart variability changes in response to performing certain types of activities. Further, the disclosed technology can generate visualizations such as charts and graphs that can accompany the analysis and support the explanations. For example, the disclosed technology can generate a line chart that shows trends in a user's heart rate over time. As such, the disclosed technology allows for improved processing of health data such that a user can receive an actionable analysis of the user's personal health data based on specific user queries. The disclosed technology therefore generates natural language explanations and visualizations of significant statistical relationships in a format that facilitates understanding by a user.

Accordingly, the disclosed technology can generate improved analyses and visualizations that are based on specific user queries. Further, the disclosed technology can assist a user in more effectively and/or safely performing the technical task of health data processing by means of a continued and/or guided human-machine interaction process in which queries are received and the disclosed technology generates real-time analyses and visualizations based on continuously updated health data. The disclosed technology allows for the generation of personalized analyses that better address a particular user's queries.

The disclosed technology can be implemented in a computing system (e.g., a health data computing system) that is configured to access data and/or perform operations on the data. For example, the operations performed by the computing system can comprise receiving and/or processing queries, determining topics of the queries, determining key metrics of health data, determining analytical techniques based on the topics and key metrics, determining analytical results, generating an analysis comprising explanations of the analytical results, and generating visualizations based on the analysis. Further, the computing system can leverage one or more machine-learned models that have been configured and/or trained to generate outputs that can comprise topics of queries, key metrics of health data, analytical techniques based on the topics and key metrics, analytical results, an analysis comprising explanations of the analytical results, and/or visualizations based on the analysis comprising the explanations.

The computing system can be included in a wearable computing device (e.g., a smartwatch or smart band), mobile device (e.g., a smartphone or laptop computing device), and/or as part of a system that includes a server computing device that receives data associated with a queries about a user's health data from a user's client computing device (e.g., the user's smartwatch and/or smart phone), performs operations based on the data and sends output comprising an analysis and visualizations associated with the user's queries and health data back to the client computing device. In some embodiments, the computing system can include specialized hardware and/or software that enables the performance of operations specific to the disclosed technology. For example, the computing system can include one or more application specific integrated circuits that are configured to perform operations associated with the generation of health analyses and visualizations that can assist a user in the task of processing health data.

The computing system can receive, access, and/or retrieve one or more queries. For example, the computing system can receive one or more queries via a user interface of a wearable device (e.g., a smartwatch) or a mobile device (e.g., a smartphone or laptop computing device). In some embodiments, the one or more queries can be received via a chat interface that is configured to receive text-based queries and/or audio-based queries. For example, the chat interface can be implemented on a smartphone or wearable device (e.g., smartwatch) that accepts text-based queries via tactile inputs to a touchscreen of a smartphone and/or audio-based queries via a voice input to a microphone of a smartwatch.

The one or more queries can be associated with health data (e.g., a request for information associated with health data of a user) comprising health information (e.g., health information associated with a user that sent the one or more queries). For example, the health data can comprise data associated with a user's physical state (e.g., a user's heart rates at one or more time intervals, a user's gender, a user's age, and/or a user's mass at one or more time intervals), data associated with a user's activities (e.g., kilometers rowed per week or steps taken daily), data associated with a user's nutrition (e.g., the types and amounts of food a user consumes daily), and/or other data associated with the user (e.g., a user's name and/or information added to the health data by a user such as activity goals or fitness goals).

In some embodiments, one or more security measures can be implemented to ensure that the one or more queries are from the particular user that is associated with the health data. For example, passcode or fingerprint authentication may be used to determine that the identity of the user associated with the query matches the identity of the user associated with the health data. Further, the health data can be encrypted to secure the health data from unauthorized access.

The health data can comprise activity information associated with one or more activities performed by a user. Further, the activity information can comprise one or more times at which the one or more times at which the one or more activities are performed (e.g., the times at which a user runs and/or swims). For example, the health data can comprise the types of activities a user performs (e.g., sleeping, walking, running, rowing, cycling, and/or swimming), an amount of time a user performs an activity (e.g., weekly hours spent running or walking), an estimated number of calories that are expended to perform an activity (e.g., 1200 kcal expended after rowing for an hour). Further, the health data can comprise a user's activity goals (e.g., a goal to row more than a threshold distance every week or walk more than a threshold number of steps daily).

The health data can comprise nutritional information associated with food consumption (e.g., one or more foods consumed by a user). For example, the health data can comprise mealtimes, the types of foods consumed by a user (e.g., rice, bread, fish, vegetables, meat, fruit, and/or dishes such as borsch, dumplings, or lasagna), an estimated number of proteins consumed per meal, an estimated number of fat consumed per meal, an estimated number of carbohydrates consumed per meal, an estimated caloric intake of a user, and/or the times at which a user consumes water or other liquids. Further, the health data can comprise a user's nutritional goals (e.g., a goal to eat vegetables or fruits daily or to keep caloric intake below some threshold amount).

The computing system can determine and/or generate one or more topics of the one or more queries, one or more key metrics (e.g., one or more key metrics of the health data), and/or one or more analytical techniques. The one or more analytical techniques can be based on the one or more topics and/or the one or more key metrics. Determining and/or generating the one or more topics, one or more key metrics, and/or one or more analytical techniques can be based on inputting the one or more queries and/or the health data into one or more machine-learned models. The one or more machine-learned models can comprise one or more large language models (LLMs) that are configured and/or trained to process (e.g., parse) the one or more queries and/or determine the one or more topics, the one or more key metrics, and/or the one or more analytical techniques.

Further, the computing system can leverage the capabilities of these machine-learned models to ascertain the user's intent as expressed in the one or more queries. For example, a computing system can process a query to identify various topics that can include relationships between different health metrics, the achievement or progression towards one or more health or activity goals, the identification of trends in health data over a specific period, the detection of anomalies within the health data, and/or comparisons of a user's health metrics to broader health standards or aggregated data sets. A computing system can be configured to derive these topics by analyzing the phrasing, keywords, and/or contextual cues present in the one or more queries.

Based on determining one or more topics, the computing system can identify one or more key metrics from the available health data that are particularly relevant to the determined topics. The key metrics can encompass a wide range of specific quantitative or qualitative health data points. For instance, the key metrics can include heart rates (e.g., resting heart rate, maximum heart rate, heart rate variability) at various times, oxygen saturation levels at specific times, breathing rate data, blood pressure readings at one or more times, skin temperature measurements, estimated caloric intake over defined periods (e.g., daily caloric intake), sleep duration (e.g., nightly sleep duration), bedtime information, body mass, distance travelled (e.g., daily running distances), and/or step counts. Additionally, the key metrics can comprise statistical derivatives of these data points, comprising one or more averages (e.g., a mean or median), one or more modes, variances, or standard deviations over particular time intervals (e.g., an average heart rate or sleep duration over a week). The selection of these key metrics can be guided by the determined topics, ensuring that the subsequent analysis focuses on the most pertinent data points that can address the user's query.

Furthermore, the computing system can determine one or more analytical techniques suitable for processing the identified key metrics in light of the one or more topics. The one or more analytical techniques can be selected from a plurality of statistical analysis techniques and can include operations comprising comparing one or more key metrics at a first time interval to the same or different key metrics at a second time interval (e.g., comparing current heart rate variability to heart rate variability from two months prior). Other analytical techniques can include comparing one or more key metrics of a user to one or more key metrics derived from aggregate health data of other users (e.g., comparing a user's resting heart rate to resting heart rates of other users within a similar demographic). The analytical techniques can also comprise determining one or more mean values, determining one or more standard deviations, determining one or more correlations between two or more key metrics, and/or performing one or more regression analyses (e.g., linear regression analysis) on at least one key metric.

The machine-learned models, particularly the one or more large language models (LLMs), can be configured and/or trained to facilitate this process. Their configuration and/or training can enable them to effectively parse the linguistic structure and semantic content of the one or more queries, thereby extracting the underlying topics. Subsequently, these models can map the identified topics to specific key metrics present in the health data. Moreover, various query types, health data sets, and/or corresponding analytical methods can be used to configure and/or train the machine-learned to determine and/or select one or more analytical techniques that align with the identified topics and/or key metrics. This capability allows the computing system to provide a relevant and targeted analysis of the health data.

The one or more machine-learned models can determine one or more topics which can comprise relationships between metrics indicated in health data (e.g., relationships such as correlations between sleep patterns and nutrition), achievement of goals (e.g., achievement of weight loss goals), identification of trends (e.g., trends such as decreases in heart rates over time or increases in running distances over time), identification of anomalies in health data, and/or comparisons of the user to health standards (e.g., comparison of a user's health metrics to aggregate health data which can comprise the aggregated health metrics of millions of users or a comparison of a user's current health metrics to previous health metrics of the user).

The one or more machine-learned models can determine one or more key metrics which can comprise one or more heart rates at one or more times, one or more resting heart rates at one or more times, a maximum heart rate (MHR), resting heart rate (RHR) at one or more times, heart rate variability (HRV) at one or more times, oxygen saturation (SpO₂) at one or more times, breathing rate, blood pressure at one or more times, skin temperature at one or more times, caloric intake (e.g., daily caloric intake) at one or more times, sleep duration at one or more times (e.g., nightly sleep duration), bedtime at one or more times (e.g., daily bedtimes), mass (e.g., body mass in kilograms) at one or more times, distance travelled at one or more times (e.g., daily running distances), and/or one or more step counts (e.g., daily step count). Further, the key metrics can comprise one or more averages (mean or median), one or more modes, variances, standard deviations. For example, the key metrics can comprise an average heart rate or sleep duration over some time interval (e.g., a week).

The one or more machine-learned models can determine the one or more analytical techniques based on processing input comprising the one or more topics, the health data, and/or the one or more key metrics. Further, determining the one or more analytical techniques can be based on the one or more machine-learned models being configured and/or trained to determine certain analytical techniques (e.g., statistical analysis techniques) based on the accurate determination of similar analytical techniques for similar topics and/or key metrics during training of the one or more machine-learned models. For example, the one or more machine-learned models can select a first type of analytical technique for a first topic (e.g., achieving running distance goals) and first key metric (e.g., running times) based on the one or more machine-learned models being configured and/or trained to select the first type of analytical technique for a second topic that is the same as the first topic (e.g., achieving distance goals) and a second key metric (e.g., rowing times) that is different from the first key metric (e.g., rowing times instead of running times).

The one or more analytical techniques can comprise comparing one or more key metrics at one time interval to one or more key metrics at a different time interval (e.g., comparing current heart rate variability to heart rate variability two months ago), comparing one or more key metrics of one user to one or more key metrics of an aggregation of other users (e.g., comparing a user's resting heart rate to the resting heart rates of other users in the same demographic as the user), determining one or more mean values of at least one key metric of the one or more key metrics (e.g., mean step counts over a three-month time interval), determining a standard deviation associated with at least one key metric of the one or more key metrics, determining one or more correlations between two or more key metrics of the one or more key metrics, and/or performing one or more regression analyses (e.g., linear regression analysis) on at least one key metric of the one or more key metrics.

Determining the one or more analytical techniques can comprise selecting, based on the one or more topics and/or the one or more key metrics, the one or more analytical techniques from a plurality of statistical analysis techniques. For example, the one or more machine-learned models can be configured and/or trained to evaluate the one or more topics and the one or more key metrics and generate output identifying one or more statistical analysis techniques that are selected from a template comprising a plurality of statistical analysis techniques. The one or more machine-learned models can be configured and/or trained to select a statistical analysis technique that is most relevant to the one or more topics and/or key metrics.

In some embodiments, the one or more machine-learned models can be configured to determine a range of dates from which the one or more key metrics are selected. For example, if the one or more queries indicate “TELL ME HOW MUCH I ATE LAST WEEK” the one or more machine-learned models can determine the dates of the last week and that the one or more key metrics that are associated with food consumption may be selected from a time interval that includes those dates.

The computing system can determine and/or generate one or more analytical results. The one or more analytical results can be based on performing the one or more analytical techniques on at least the health data comprising the one or more key metrics. Further, the one or more analytical results can comprise one or more statistical relationships between at least one of the one or more key metrics (e.g., one or more key metrics of the user that generated the one or more queries) and at least one of the one or more key metrics based on aggregate health data (e.g., aggregate health data based on millions of other users such as the average resting heart rates of millions of users in a particular age range). For example, if the one or more key metrics comprise a user's heart rates and sleep durations over a two-month time interval and the one or more analytical techniques are directed at determining whether there is a correlation between the heart rates and sleep durations, the one or more analytical results can comprise a correlation coefficient that indicates the strength of the relationship between the heart rates and sleep durations over the two-month time interval.

In some embodiments, determining the one or more analytical results can comprise inputting the health data comprising the one or more key metrics into one or more machine-learned models that are configured and/or trained to determine and/or generate the one or more analytical results. The one or more machine-learned models can be configured and/or trained to perform operations to determine the one or more analytical results based on the one or more key metrics and/or the one or more analytic techniques. For example, the one or more machine-learned models can be configured and/or trained to determine the one or more analytical results until the output generated by the one or more machine-learned models exceeds some accuracy threshold.

The computing system can generate one or more analyses (e.g., an analysis) that can comprise one or more explanations of the one or more analytical results. Generation of the analysis can be based on inputting the one or more analytical results into one or more machine-learned models that are configured and/or trained to generate the one or more explanations. The one or more explanations can comprise one or more natural language explanations of the one or more analytical results.

Generating the one or more explanations can comprise generating a technical interpretation and/or transformation based on the one or more analytical results (e.g., quantitative analytical results) that is in a readily understandable format. Further, the one or more analytical results, which can comprise numerical outputs from statistical operations (e.g., correlation coefficients, mean values, standard deviations, and/or regression parameters), can be provided as input to the one or more machine-learned models. The one or more machine-learned models, which can include large language models (LLMs) configured for natural language generation, can be precisely structured and trained to process such data. The one or more machine-learned models can be configured and/or trained to determine the significance of the one or more analytical results, to identify one or more patterns, and/or to generate one or more explanations that articulate one or more underlying technical relationships within the health data.

The one or more machine-learned models can interpret the output from the analytical techniques and generate a coherent explanation. For example, if an analytical technique determines a strong negative correlation between a user's average nightly sleep duration and their resting heart rate, the models can be capable of generating an explanation such as, “A CONSISTENT TREND INDICATES THAT AS YOUR AVERAGE NIGHTLY SLEEP DURATION INCREASES, YOUR RESTING HEART RATE TENDS TO DECREASE, WHICH CAN SIGNIFY IMPROVED CARDIOVASCULAR RECOVERY.”

Furthermore, the natural language explanations can provide context by referencing comparative data or trends over time. The natural language explanation can comprise an indication of the result and also provide a temporal comparison, which can enhance the technical understanding of the progression. For example, if an analytical result indicates that a user's daily step count has increased significantly over the past three months, a generated explanation may state, “YOUR DAILY STEP COUNT HAS SHOWN A SUBSTANTIAL UPWARD TREND OVER THE LAST THREE MONTHS, REACHING LEVELS APPROXIMATELY 20% HIGHER THAN YOUR ACTIVITY FROM THE PRECEDING QUARTER.”

The one or more explanations can also address the relevance of a user's health metrics in relation to aggregated health data from other users, where relevant comparative demographics can be determined. For example, if an analytical result indicates that a user's blood pressure is lower than the average for individuals within a similar age group and activity level, a corresponding explanation can indicate, “YOUR BLOOD PRESSURE READINGS CONSISTENTLY FALL BELOW THE AVERAGE FOR INDIVIDUALS IN YOUR DEMOGRAPHIC GROUP, SUGGESTING POTENTIALLY ROBUST CARDIOVASCULAR HEALTH RELATIVE TO A PEER POPULATION.”

In some embodiments, generating the one or more explanations can comprise identifying and verbalizing deviations or anomalies present in the health data. If an analytical result flags an unusual spike in skin temperature, the explanation can highlight this anomaly and suggest factors that may be associated with it. For example, an explanation can indicate, “AN UNUSUAL ELEVATION IN YOUR SKIN TEMPERATURE WAS NOTED ON FEBRUARY 5TH, WHICH MAY BE CORRELATED WITH THE INCREASE IN THE FREQUENCY AND INTENSITY OF YOUR EXERCISE SESSIONS.”

The analysis can comprise one or more recommendations based on the one or more analytical results. Further, the one or more recommendations can comprise one or more natural language recommendations based on at least one of the one or more key metrics. For example, the analysis can comprise recommendations that are based on the analysis such as a recommendation focused on a key metric (e.g., sleep duration) that indicates that sleeping more may be beneficial to a user if the health data indicates that a user sleeps less than a threshold amount (e.g., less than eight hours per night).

Generating an analysis can comprise determining one or more demographics that correspond to the health data (e.g., a demographic associated with the health data of the user that sent the one or more queries). For example, the health data can indicate that a user is a thirty-year old woman with a resting heart rate below 50 beats per minute.

Further, generating the analysis can comprise generating one or more explanations comprising one or more comparisons of the one or more key metrics (e.g., one or more key metrics of the user that sent the one or more queries) to the one or more key metrics of aggregate health data that corresponds to the one or more demographics. The analysis that is generated may comprise one or more comparisons of the health data associated with the user that sent the one or more queries to one or more key metrics (e.g., the same one or more key metrics as the user that sent the one or more queries) of aggregate health data which can be based on the health data of other users (e.g., average resting heart rates for thirty-year old women). In some embodiments, the one or more demographics can comprise ranges associated with the one or more key metrics. For example, an age range may comprise a 30-35 year-old age range or a 40-50 year-old age range. Further, a resting heart rate range may comprise a 50-55 beats per minute heart rate range or a 56-60 beats per minute heart rate range.

Further, the generation of the one or more recommendations can be context-dependent and can leverage additional information including a user's historical health data, activity goals, and/or nutritional patterns. For example, if an analytical result shows a trend of increasing body mass while caloric intake remains constant and activity levels decrease, the computing system can generate one or more recommendations that address the interplay of these key metrics. For example, the computing system can generate the recommendation “YOUR BODY MASS HAS SHOWN AN UPWARD TREND WHILE YOUR ACTIVITY LEVELS HAVE DECREASED. ADJUSTING DAILY CALORIC INTAKE TO ALIGN WITH CURRENT ACTIVITY LEVELS MAY ASSIST IN MODULATING BODY MASS METRICS.” This technical guidance combines multiple data points to offer a multifaceted approach to influencing physiological parameters.

The one or more recommendations can also be generated based on one or more comparisons to aggregated health data sets corresponding to similar demographics. This type of recommendation can provide a comparative technical benchmark and propose an adjustment aimed at bringing a specific metric within a desired range. For example, if a user's estimated caloric intake significantly deviates from a recommended range for their age and activity level, a recommendation could highlight this discrepancy and suggest adjustments. For example, the computing system can generate the recommendation indicating “YOUR ESTIMATED DAILY CALORIC INTAKE IS PRESENTLY BELOW THE AVERAGE RANGE FOR INDIVIDUALS OF YOUR AGE AND ACTIVITY LEVEL, WHICH MAY IMPACT ENERGY METRICS. GRADUALLY INCREASING YOUR CALORIC INTAKE TOWARDS A RECOMMENDED BASELINE MAY HELP MAINTAIN ENERGY LEVELS.”

The computing system can generate one or more visualizations. The one or more visualizations can be based on the analysis (e.g., the analysis comprising the one or more explanations). The one or more visualizations can graphically convey the one or more analytical results and facilitate the technical comprehension of complex health data patterns and/or relationships. The one or more visualizations can include text (e.g., words, symbols, and/or numbers) and/or images that can be generated and outputted to a display device (e.g., a display device of a smartphone or fitness tracker). Further, the one or more visualizations can comprise one or more charts (e.g., area charts, pie charts, bar charts, line charts, punchcard charts, and/or scatter plots), one or more graphs, one or more heatmaps, one or more histograms, and/or one or more infographics that can include a combination of text and images. By way of further example, the computing system can generate a bubble chart to illustrate the relationship between key metrics (e.g., body mass, daily caloric intake, and average resting heart rate), by representing each data point as a bubble with its size correlating to a third metric. Further, the computing system can generate a gauge chart which can depict progression towards a specific physiological goal (e.g., a target heart rate zone or a daily step count objective).

Further, the computing system may generate box plots to display the distribution characteristics of various key metrics over a defined period, showing median values, quartiles, and/or outliers for one or more metrics (e.g., daily sleep duration and/or blood pressure readings). This visual representation can assist in identifying the variability and spread of the data. The one or more visualizations can also comprise network graphs to illustrate complex interdependencies and/or correlations between multiple health parameters, such as the relationship between sleep quality, activity levels, and stress markers. A network graph can depict nodes that represent metrics, and edges that indicate the strength or type of the statistical association between nodes. In some embodiments, a computing system can be configured to generate one or more visualizations comprising a dashboard visualization. The dashboard visualization can comprise one or more charts and/or textual explanations into a unified display, thereby offering a comprehensive technical overview of a user's health profile and analytical findings.

Generating the one or more visualizations can comprise generating one or more charts corresponding to the one or more explanations. For example, one or more visualizations of an analysis of changes in a user's heart rate variability over a two-month time interval can comprise a line chart that shows the daily changes in the user's heart rate variability over the two-month time interval.

Generating the one or more visualizations can comprise inputting the one or more explanations into one or more machine-learned models that are configured and/or trained to generate the one or more visualizations based on input comprising the one or more explanations. For example, generating the one or more visualizations can be based on the one or more machine-learned models being configured and/or trained to generate certain visualizations (e.g., line graphs) based on the accurate generation of similar visualizations for similar analyses and/or explanations during training of the one or more machine-learned models. For example, the one or more machine-learned models can generate a first type of visualization (e.g., a line chart) for a first analysis (e.g., an analysis of a user's running performance over time) based on the one or more machine-learned models being configured and/or trained to generate the first type of visualization (e.g., a line chart) for a second analysis that is different from the first analysis (e.g., swimming performance analysis instead of running performance analysis) but in the same class as the first analysis (e.g., athletic performance over time).

The systems, methods, devices, computer-readable media (e.g., tangible non-transitory computer-readable media) in the disclosed technology can provide a variety of technical effects and benefits including an improvement in the generation of helpful analyses of health data that are personalized based on the queries of a user. In particular, the disclosed technology may assist a user (e.g., a user of a fitness tracking device) in performing technical tasks by means of a continued and/or guided human-machine interaction process in which health data can be continuously updated and queried in order to generate analyses that can include explanations of relationships between key health metrics.

Further, the disclosed technology can provide the technical effect of improving the effectiveness with which health related tasks are performed. For example, the computing system can be continuously updated based on user queries and/or changes in the user's health data, thereby allowing for more timely provision of relevant personalized health-related information to a user. For example, machine-learned models that are used as part of the process of generating personalized health analyses can be continuously trained and/or updated in response to updated health data that is specific to the user. This can have the effect of providing more relevant and/or accurate analyses based on a particular user's health data. Further, the disclosed technology can generate visualizations that can support the health analyses and facilitate a user's understanding of potentially complex trends and/or relationships in health data.

Further, the disclosed technology can provide the technical effect of improving resource utilization within a computing system. For example, by precisely determining the one or more topics and/or the one or more key metrics from a user's query, the computing system can facilitate more targeted data retrieval and processing, which can reduce the computational overhead associated with generating the analyses. This can lead to decreased demand on processor cycles and memory resources. A more focused analysis can also enable reduced data transmission volumes across networks, as only pertinent health data may be communicated to the processing components, thereby optimizing network bandwidth utilization. Additionally, such efficiencies can contribute to a reduction in the power consumption of a health processing device, which can extend battery life for portable or wearable devices engaged in continuous health monitoring and analysis.

Additionally, the disclosed technology can enhance the adaptability and/or robustness of health data processing systems. For example, the continuous training and updating of the one or more machine-learned models can allow the system to adapt to evolving health data patterns, new types of user queries, and/or changes in aggregated health data, which can increase the long-term reliability and accuracy of the generated analyses. This technical adaptability can also facilitate the scalable integration of diverse health data sources, such as additional sensor types or external health records, into a unified analytical framework without requiring substantial redesign of underlying processing logic. The system can thereby more robustly identify subtle health trends or anomalies that might otherwise be overlooked by less adaptive or less continuously updated analytical frameworks.

The disclosed technology can improve the operation of a health processing device by more effectively performing a variety of tasks with the specific benefits of improving health outcomes and/or identifying potentially positive or potentially adverse trends in a user's health data. Further, the specific benefits provided to users can be used to improve the effectiveness of a wide range of devices and services including health monitoring devices (e.g., fitness bands) and health monitoring services. Accordingly, the improvements offered by the disclosed technology can result in tangible benefits to a variety of devices and/or systems comprising computing systems, electronic systems, and/or mechanical systems associated with processing health data.

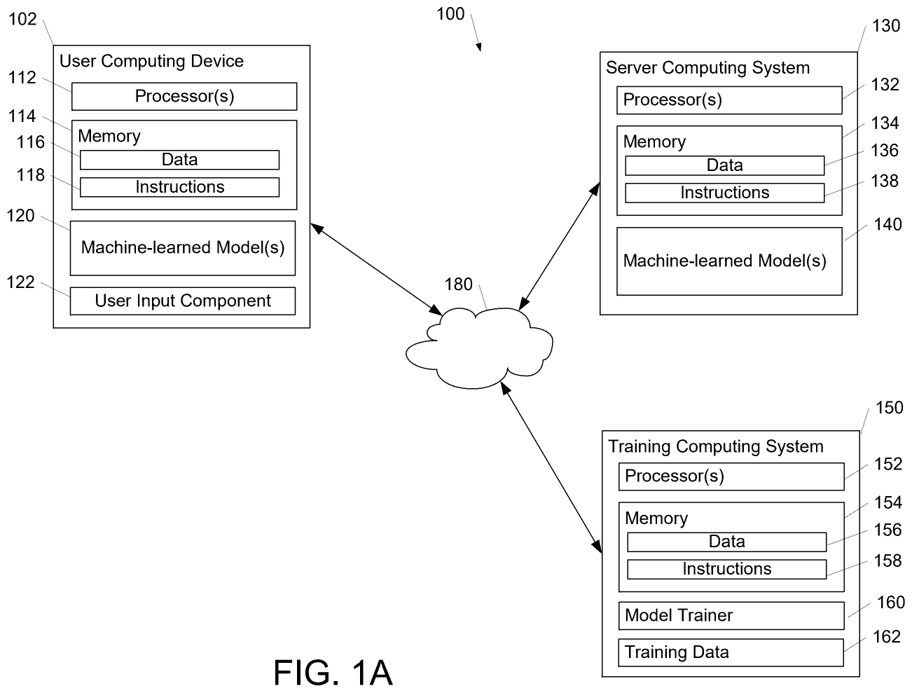

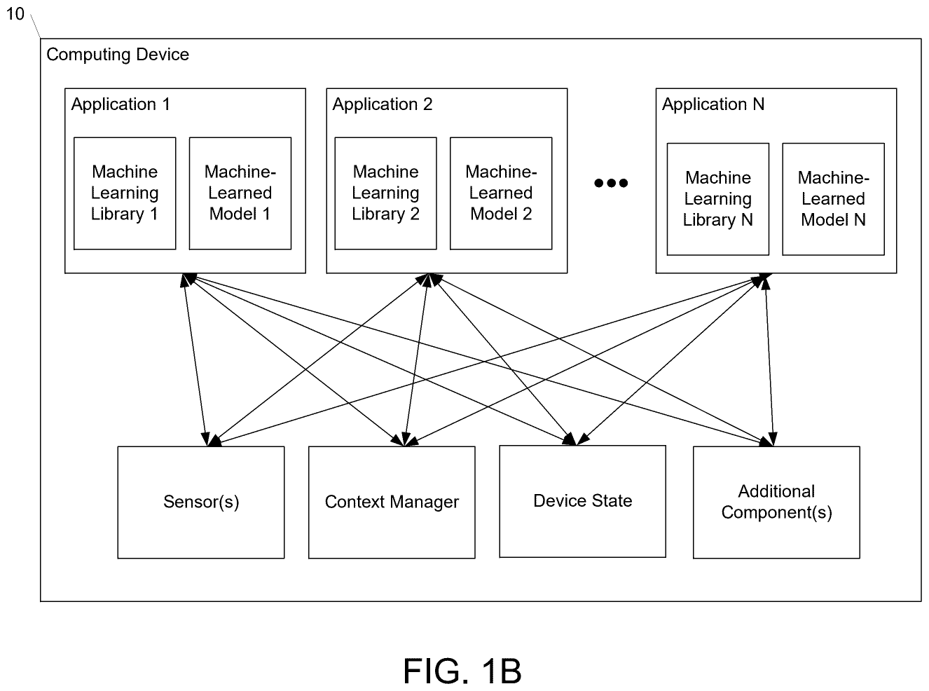

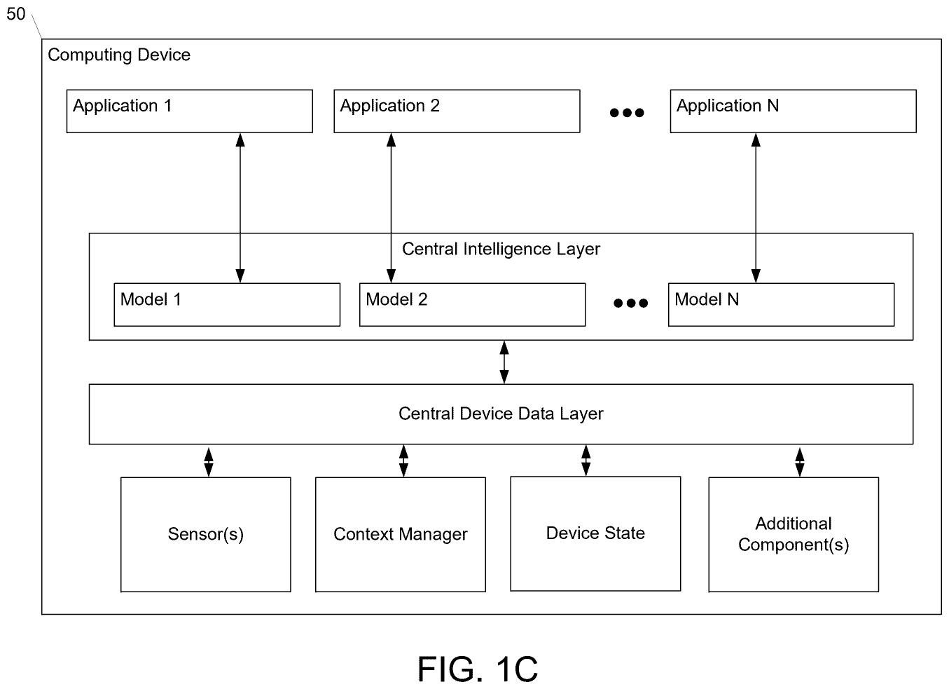

With reference now to the figures, example embodiments of the present disclosure will be discussed in further detail. FIG. 1A depicts a block diagram of an example of a computing system that generates health analyses and visualizations according to example embodiments of the present disclosure. System 100 includes a computing device 102, a server computing system 130, and a training computing system 150 that are communicatively coupled over a network 180.

The computing device 102 can comprise any type of computing device, such as, for example, a wearable computing device (e.g., a smartwatch), a personal computing device (e.g., laptop or desktop), a mobile computing device (e.g., smartphone or tablet), a gaming console or controller, an embedded computing device, or any other type of computing device.

The computing device 102 includes one or more processors 112 and a memory 114. The one or more processors 112 can be any suitable processing device (e.g., a processor core, a microprocessor, an ASIC, an FPGA, a controller, and/or a microcontroller) and can be one processor or a plurality of processors that are operatively connected. The memory 114 can include one or more non-transitory computer-readable storage media, such as RAM, ROM, EEPROM, EPROM, flash memory devices, magnetic disks, and/or combinations thereof. The memory 114 can store data 116 and instructions 118 which are executed by the processor 112 to cause the computing device 102 to perform operations.

In some implementations, the computing device 102 can store or include one or more machine-learned models 120. For example, the one or more machine-learned models 120 can be or can otherwise include various machine-learned models such as neural networks (e.g., deep neural networks) or other types of machine-learned models, comprising non-linear models and/or linear models. Neural networks can include feed-forward neural networks, recurrent neural networks (e.g., long short-term memory recurrent neural networks), convolutional neural networks or other forms of neural networks. Some example machine-learned models can leverage an attention mechanism such as self-attention. For example, some example machine-learned models can include multi-headed self-attention models (e.g., transformer models). Examples of one or more machine-learned models 120 are discussed with reference to FIGS. 1-12.

In some implementations, the one or more machine-learned models 120 can be received from the server computing system 130 over network 180, stored in the memory 114, and then used or otherwise implemented by the one or more processors 112. In some implementations, the computing device 102 can implement multiple parallel instances of a single machine-learned model of the one or more machine-learned models 120 (e.g., to perform parallel recommendation generation operations across multiple instances of the one or more machine-learned models 120).

More particularly, the one or more machine-learned models 120 can comprise one or more machine-learned models (e.g., one or more LLMs) that are configured and/or trained to parse queries (e.g., text-based queries and/or audio-based queries) determine topics of the queries, determine key metrics of health data, determine analytical techniques based on the topics and key metrics, determine analytical results, generate an analysis comprising explanations of the analytical results, and generate visualizations based on the analysis comprising the explanations.

Additionally or alternatively, one or more machine-learned models 140 can be included in or otherwise stored and implemented by the server computing system 130 that communicates with the computing device 102 according to a client-server relationship. For example, the one or more machine-learned models 140 can be implemented by the server computing system 130 as a portion of a web service (e.g., a health data processing and analysis service). Thus, one or more machine-learned models 120 can be stored and implemented at the computing device 102 and/or one or more machine-learned models 140 can be stored and implemented at the server computing system 130.

The computing device 102 can also include one or more user input components 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, an FPGA, a controller, and/or a microcontroller) and can be one processor or a plurality of processors that are operatively connected. The memory 134 can include one or more non-transitory computer-readable storage media, such as RAM, ROM, EEPROM, EPROM, flash memory devices, magnetic disks, and/or combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned models 140. For example, the one or more machine-learned models 140 can be or can otherwise include various machine-learned models. Example machine-learned models include neural networks or other multi-layer non-linear models. Example neural networks include feed forward neural networks, deep neural networks, recurrent neural networks, and convolutional neural networks. Some example machine-learned models can leverage an attention mechanism such as self-attention. For example, some example machine-learned models can include multi-headed self-attention models (e.g., transformer models). Examples of one or more machine-learned models 140 are discussed with reference to FIGS. 1-12.

The computing device 102 and/or the server computing system 130 can train the one or more machine-learned models 120 and/or the one or more machine-learned models 140 via interaction with the training computing system 150 that can be communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, an FPGA, a controller, and/or a microcontroller) and can be one processor or a plurality of processors that are operatively connected. The memory 154 can include one or more non-transitory computer-readable storage media, such as RAM, ROM, EEPROM, EPROM, flash memory devices, magnetic disks, and/or combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the one or more machine-learned models 120 and/or the one or more machine-learned models 140 stored at the computing device 102 and/or the server computing system 130 using various training or learning techniques (e.g., machine-learning techniques), such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a plurality of training iterations.

In some implementations, performing backwards propagation of errors can include performing truncated backpropagation through time. The model trainer 160 can perform a number of generalization techniques (e.g., weight decays, dropouts, and/or other generalization techniques) to improve the generalization capability of the models being trained.

In particular, the model trainer 160 can train the one or more machine-learned models 120 and/or the one or more machine-learned models 140 based on a set of training data 162. The training data 162 can include various types of data. For example, the training data 162 can include health data and/or other data that is associated with the health of one or more users, nutrition of one or more users, activities (e.g., exercise and fitness activities) of one or more users, sensor data based on one or more physical states of one or more users (e.g., heart rates). For example, the training data 162 can comprise heart rate data (e.g., the heart rate of a user at one or more times), mass data (e.g., the mass in kilograms of a user at one or more times), age data (e.g., the age of a user), blood pressure data (e.g., the blood pressure of a user at one or more times), nutrition data (e.g., the caloric intake of a user, the times at which a user cats, and/or the types of food a user cats), sleep data (e.g., the nightly sleep duration of a user at one or more times and/or the bedtimes of a user), and/or activity data (e.g., the types of activities a user performs and/or the one or more times at which a user performs a fitness activity such as running, walking, and/or rowing). Further, the training data 162 can include various publications (e.g., books, articles, and/or journals) that can be received from a variety of sources including libraries, the Internet (e.g., websites), and/or devices that can comprise sensors and can be configured to generate and/or receive data (e.g., smartwatches, smartphones, and/or fitness trackers that can be configured to receive sensor data and/or data entered by a user). The model trainer 160 can train and/or retrain the one or more machine-learned models 120 and/or the one or more machine-learned models 140 based on additional data from the training data 162 which can comprise additional health data (e.g., updated health data), new types of health data (e.g., new types of health data based on sensor data from new sensor types), and/or one or more modifications to existing health data.

In some implementations, if the user has provided consent (e.g., the user provides affirmative consent for another party to use the user's health data), the training examples can be provided by the computing device 102. Thus, in such implementations, the one or more machine-learned models 120 provided to the computing device 102 can be trained by the training computing system 150 on user-specific data received from the computing device 102. In some instances, this process can be referred to as personalizing the model.

The model trainer 160 includes computer logic utilized to provide desired functionality. The model trainer 160 can be implemented in hardware, firmware, and/or software controlling a general-purpose processor. For example, in some implementations, the model trainer 160 includes program files stored on a storage device, loaded into a memory, and executed by one or more processors. In other implementations, the model trainer 160 includes one or more sets of computer-executable instructions that are stored in a tangible computer-readable storage medium such as RAM, hard disk, or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The machine-learned models described in this specification may be used in a variety of tasks, applications, and/or use cases. In some implementations, the input to the machine-learned model(s) of the present disclosure can be text or natural language data. The machine-learned model(s) can process the text or natural language data to generate an output (e.g., based on inputting queries from a user the machine-learned model(s) can process and generate an analysis comprising one or more explanations and visualizations associated with the queries and health data of the user). As an example, the machine-learned model(s) can process the natural language data to generate a language encoding output. As another example, the machine-learned model(s) can process the text or natural language data to generate a latent text embedding output. As another example, the machine-learned model(s) can process the text or natural language data to generate a translation output. As another example, the machine-learned model(s) can process the text or natural language data to generate a classification output. As another example, the machine-learned model(s) can process the text or natural language data to generate a textual segmentation output. As another example, the machine-learned model(s) can process the text or natural language data to generate a semantic intent output. As another example, the machine-learned model(s) can process the text or natural language data to generate an upscaled text or natural language output (e.g., text or natural language data that is higher quality than the input text or natural language). As another example, the machine-learned model(s) can process the text or natural language data to generate a prediction output.

In some implementations, the input to the machine-learned model(s) of the present disclosure can be speech data. The machine-learned model(s) can process the speech data to generate an output. As an example, the machine-learned model(s) can process the speech data to generate a speech recognition output. As another example, the machine-learned model(s) can process the speech data to generate a speech translation output. As another example, the machine-learned model(s) can process the speech data to generate a latent embedding output. As another example, the machine-learned model(s) can process the speech data to generate an encoded speech output (e.g., an encoded and/or compressed representation of the speech data). As another example, the machine-learned model(s) can process the speech data to generate an upscaled speech output (e.g., speech data that is higher quality than the input speech data). As another example, the machine-learned model(s) can process the speech data to generate a textual representation output (e.g., a textual representation of the input speech data). As another example, the machine-learned model(s) can process the speech data to generate a prediction output.

In some implementations, the input to the machine-learned model(s) of the present disclosure can be latent encoding data (e.g., a latent space representation of an input). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a search output. As another example, the machine-learned model(s) can process the latent encoding data to generate a reclustering output. As another example, the machine-learned model(s) can process the latent encoding data to generate a prediction output.

In some implementations, the input to the machine-learned model(s) of the present disclosure can be statistical data. Statistical data can be, represent, or otherwise include data computed and/or calculated from some other data source. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the machine-learned model(s) can be configured to perform a task that includes encoding input data for reliable and/or efficient transmission or storage (and/or corresponding decoding). For example, the task may be an audio compression task. The input may include audio data and the output may comprise compressed audio data. In another example, the input includes visual data (e.g., one or more images or videos), the output comprises compressed visual data, and the task is a visual data compression task. In another example, the task may comprise generating an embedding for input data (e.g., input audio data or visual data).

In some cases, the input includes audio data representing a spoken utterance and the task is a speech recognition task. The output may comprise a text output which is mapped to the spoken utterance. In some cases, the task comprises encrypting or decrypting input data. In some cases, the task comprises a microprocessor performance task, such as branch prediction or memory address translation.

FIG. 1A illustrates one example computing system that can be used to implement the present disclosure. Other computing systems can be used as well. For example, in some implementations, the computing device 102 can include the model trainer 160 and the training data 162. In such implementations, the one or more machine-learned models 120 can be both trained and used locally at the computing device 102. In some of such implementations, the computing device 102 can implement the model trainer 160 to personalize the one or more machine-learned models 120 based on user-specific data.

FIG. 1B depicts a block diagram of an example of a computing device that generates health analyses and visualizations according to example embodiments of the present disclosure. A computing device 10 can be a user computing device or a server computing device.

The computing device 10 can include a number of applications (e.g., applications 1 through N). Each application contains its own machine-learned library and machine-learned model(s). For example, each application can include a machine-learned model. Example applications include a health data processing application, a statistical analysis application, a text messaging application, an email application, a dictation application, a virtual keyboard application, and/or a browser application.

As illustrated in FIG. 1B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 1C depicts a block diagram of an example computing device that generates health analyses and visualizations according to example embodiments of the present disclosure. A computing device 50 can be a user computing device or a server computing device.

The computing device 50 includes a number of applications (e.g., applications 1 through N). Each application is in communication with a central intelligence layer. Example applications include a health application (e.g., an application that is used to process health data that can be based on sensor output from a wearable computing device of a user), text messaging application, an email application, a dictation application, a virtual keyboard application, and/or a browser application. In some implementations, each application can communicate with the central intelligence layer (and model(s) stored therein) using an API (e.g., a common API across all applications).

The central intelligence layer includes a number of machine-learned models. For example, as illustrated in FIG. 1C, a respective machine-learned model can be provided for each application and managed by the central intelligence layer. In other implementations, two or more applications can share a single machine-learned model. For example, in some implementations, the central intelligence layer can provide a single model for the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 1C, the central device data layer can communicate with a number of other components of the computing device, such as, for example, one or more sensors, a context manager, a device state component, and/or additional components. In some implementations, the central device data layer can communicate with each device component using an API (e.g., a private API).

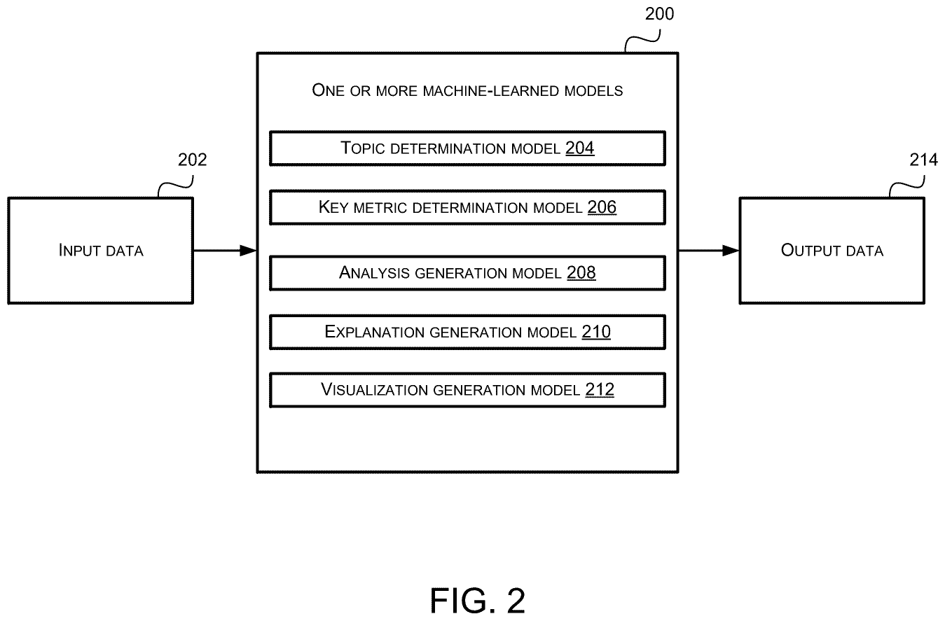

FIG. 2 depicts a block diagram of examples of machine-learned models according to example embodiments of the present disclosure. In some implementations, the one or more machine-learned models 200 can be trained to receive input data 202 that can comprise one or more queries for information associated with health data of a user. As a result of receipt of the input data 202 the one or more machine-learned models 200 can generate output data 214 that can comprise one or more topics that can be based on the one or more queries, one or more key metrics that can be based on the health data, one or more analytical techniques that can be based on the one or more topics and/or one or more key metrics, one or more analytical results that can be based on performing the one or more analytical techniques on the health data, an analysis that can comprise one or more explanations of the analytical results, and/or one or more visualizations that can be based on the analysis.

In some implementations, the one or more machine-learned models 200 can include a topic determination model 204 that is operable to generate one or more topics based on receiving input comprising one or more queries and/or health data. In some implementations, the one or more machine-learned models 200 can include a key metric determination model 206 that is operable to generate one or more key metrics based on receiving input comprising one or more queries, one or more topics, and/or health data. In some implementations, the one or more machine-learned models 200 can include an analysis generation model 208 that is operable to generate one or more analytical techniques based on the one or more topics and/or the one or more key metrics and/or one or more analytical results based on health data comprising one or more key metrics. In some implementations, the one or more machine-learned models 200 can include an explanation generation model 210 that is operable to generate an analysis comprising one or more explanations of one or more analytical results. In some implementations, the one or more machine-learned models 200 can include a visualization generation model 212 that is operable to generate one or more visualizations based on the analysis.

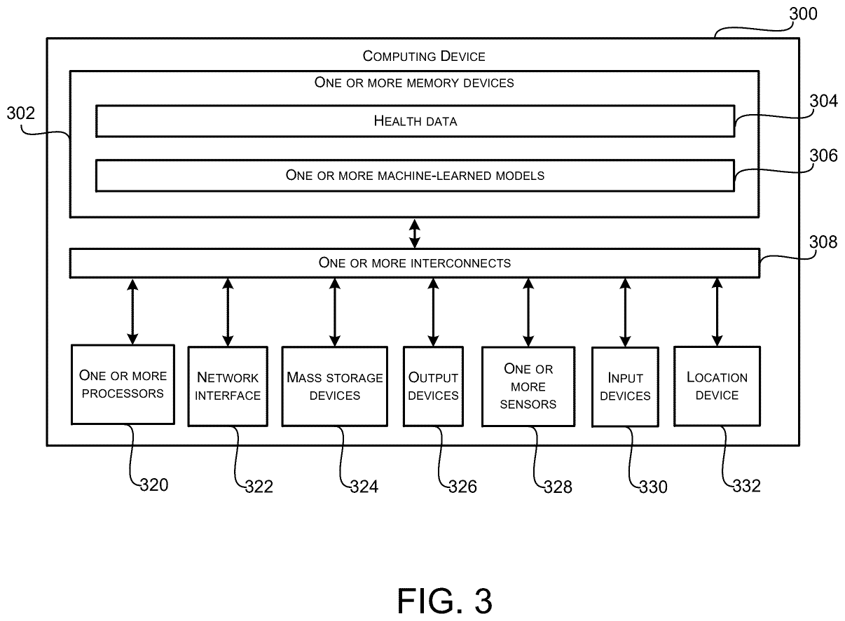

FIG. 3 depicts an example of a computing device according to example embodiments of the present disclosure. A computing device 300 can include one or more features and/or capabilities of the computing device 102, the server computing system 130, and/or the training computing system 150. Furthermore, the computing device 300 can perform one or more actions and/or operations performed by the computing device 102, the server computing system 130, and/or the training computing system 150, which are described with respect to FIG. 1A.

As shown in FIG. 3, the computing device 300 can include one or more memory devices 302, health data 304, one or more machine-learned models 306, one or more interconnects 308, one or more processors 320, a network interface 322, one or more mass storage devices 324, one or more output devices 326, one or more sensors 328, one or more input devices 330, and/or the location device 332. The computing device 300 can be configured as a wearable computing device (e.g., a smartwatch, fitness tracker, fitness band, extended reality glasses, and/or smart ring) and/or a mobile computing device (e.g., a smartphone, tablet computing device, and/or laptop computing device). Further, the computing device 300 can process and/or generate data (e.g., health data) based on one or more states of a user (e.g., one or more physical states of a user that are detected by the one or more sensors 328 of the computing device 300), data that is received from another computing device (e.g., health data that is generated by a remote computing device which can include another fitness tracking device), and/or data that is entered by a user (e.g., a user can enter information indicating the types of food a user consumes).

The one or more memory devices 302 can store information and/or data (e.g., the health data 304 and/or the one or more machine-learned models 306). Further, the one or more memory devices 302 can include one or more computer-readable mediums (e.g., tangible non-transitory computer-readable media), including RAM, ROM, EEPROM, EPROM, flash memory devices, magnetic disks, and combinations thereof. The information and/or data stored by the one or more memory devices 302 can be executed by the one or more processors 320 to cause the computing device 300 to perform operations including operations associated with receiving and/or processing queries, determining topics of the queries, determining key metrics of health data, determining analytical techniques based on the topics and key metrics, determining analytical results, generating an analysis comprising explanations of the analytical results, and generating visualizations based on the analysis comprising the explanations.

The health data 304 can include one or more portions of data (e.g., the data 116, the data 136, and/or the data 156, which are depicted in FIG. 1A) and/or instructions (e.g., the instructions 118, the instructions 138, and/or the instructions 158 which are depicted in FIG. 1A) that are stored in the memory 114, the memory 134, and/or the memory 154, respectively. Furthermore, the health data 304 can include information associated with the health and/or one or more physical states of a user (e.g., a user of the computing device 300). In some embodiments, the health data 304 can be received from one or more computing systems (e.g., the server computing system 130 that is depicted in FIG. 1) which can include one or more computing systems that are remote (e.g., in another building) from the computing device 300.

The health data 304 can include one or more portions of data (e.g., the data 116, the data 136, and/or the data 156, which are depicted in FIG. 1A) and/or instructions (e.g., the instructions 118, the instructions 138, and/or the instructions 158 which are depicted in FIG. 1A) that are stored in the memory 114, the memory 134, and/or the memory 154, respectively. Furthermore, the health data 304 can include information that was generated by the one or more sensors 328 of the computing device 300. For example, the health data 304 can comprise an indication of a number of steps detected by a motion sensor (e.g., one or more accelerometers) of the one or more sensors 328. In some embodiments, the health data 304 can be received from one or more computing systems (e.g., the server computing system 130 that is depicted in FIG. 1) which can include one or more computing systems that are remote from the computing device 300.

The one or more machine-learned models 306 (e.g., the one or more machine-learned models 120, the one or more machine-learned models 140, and/or the machine-learned models 200) can include one or more portions of the data 116, the data 136, and/or the data 156 which are depicted in FIG. 1A and/or instructions (e.g., the instructions 118, the instructions 138, and/or the instructions 158 which are depicted in FIG. 1A) that are stored in the memory 114, the memory 134, and/or the memory 154, respectively. Furthermore, the one or more machine-learned models 306 can include information associated with receiving and/or processing queries, determining topics of the queries, determining key metrics of health data, determining analytical techniques based on the topics and key metrics, determining analytical results, generating an analysis comprising explanations of the analytical results, and generating visualizations based on the analysis comprising the explanations. In some embodiments, the one or more machine-learned models 306 can be received from one or more computing systems (e.g., the server computing system 130 that is depicted in FIG. 1) which can include one or more computing systems that are remote from the computing device 300.

The one or more interconnects 308 can include one or more interconnects or buses that can be used to send and/or receive one or more signals (e.g., electronic signals) and/or data (e.g., the health data 304, and/or the one or more machine-learned models 306) between devices of the computing device 300, including the one or more memory devices 302, the one or more processors 320, the network interface 322, the one or more mass storage devices 324, the one or more output devices 326, the one or more sensors 328, and/or the one or more input devices 330. The one or more interconnects 308 can be arranged or configured in different ways, including as parallel or serial connections. Further the one or more interconnects 308 can include one or more internal buses to connect the internal components of the computing device 300; and one or more external buses used to connect the internal components of the computing device 300 to one or more external devices. By way of example, the one or more interconnects 308 can include different interfaces including Industry Standard Architecture (ISA), Extended ISA, Peripheral Components Interconnect (PCI), PCI Express, Serial AT Attachment (SATA), HyperTransport (HT), USB (Universal Serial Bus), Thunderbolt, IEEE 1394 interface (FireWire), and/or other interfaces that can be used to connect components.

The one or more processors 320 can include one or more computer processors that are configured to execute the one or more instructions stored in the one or more memory devices 302. For example, the one or more processors 320 can, for example, include one or more general purpose central processing units (CPUs), application specific integrated circuits (ASICs), and/or one or more graphics processing units (GPUs). Further, the one or more processors 320 can perform one or more actions and/or operations including one or more actions and/or operations associated with the health data 304 and/or the one or more machine-learned models 306. The one or more processors 320 can include single or multiple core devices including a microprocessor, microcontroller, integrated circuit, and/or a logic device.

The network interface 322 can support network communications. For example, the network interface 322 can support communication via networks including a local area network and/or a wide area network (e.g., the Internet). Further, the network interface 322 can be used to receive data (e.g., health data) from other computing devices. The one or more mass storage devices 324 (e.g., a hard disk drive and/or a solid-state drive) can be used to store data including the health data 304 and/or the one or more machine-learned models 306.

The one or more output devices 326 can include one or more display devices (e.g., LCD display, OLED display, Mini-LED display, microLED display, plasma display, and/or CRT display), one or more light sources (e.g., LEDs), one or more audio output devices (e.g., one or more loudspeakers), and/or one or more haptic output devices (e.g., one or more devices that are configured to generate vibratory output). For example, the one or more output devices 326 can comprise a touch sensitive display that is used to output an interface (e.g., a user interface) that can be configured to receive queries (e.g., text-based queries via the touch sensitive display) and generate an analysis (e.g., a statistical analysis of a user's health data based on the user's queries) and visualizations based on the analysis.

The one or more sensors 328 can comprise one or more heart rate sensors, one or more blood pressure sensors, one or more accelerometers, one or more gyroscopes, one or more altimeters, one or more temperature sensors (e.g., one or more thermometers), one or more bioimpedance sensors, one or more barometric pressure sensors, and/or one or more oximetry sensors. The one or more sensors 328 can be configured to generate sensor output comprising health data. For example, the one or more sensors 328 can generate health data comprising heart rate data based on sensor output from the one or more heart rate sensors. By way of further example, the one or more sensors 328 can generate health data comprising oxygen saturation (SpO₂) data based on sensor output from the one or more oximetry sensors. The one or more sensors 328 can be configured to generate health data based on data from a single sensor (e.g., an accelerometer) of the one or more sensors 328, multiple sensors of the one or more sensors 328 (e.g., an accelerometer and a gyroscope), and/or data from other devices (e.g., location data from the location device 332). For example, the one or more sensors 328 can generate health data comprising sleep metrics based on the use of location data from the location device 332 (e.g., location data that is used to indicate the location at which a user is sleeping), health data 304 (e.g., motion data that can indicate how much a user moves while sleeping) from one or more accelerometers of the one or more sensors 328, and/or the health data 304 (e.g., heart rate data that can indicate a user's heart rate when the user is asleep) from one or more heart rate monitors of the one or more sensors 328.

The one or more input devices 330 can include one or more keyboards, one or more touch sensitive devices (e.g., a touch screen display), one or more buttons (e.g., a power button and/or volume buttons), one or more microphones, and/or one or more imaging devices (e.g., one or more cameras).

The one or more memory devices 302 and the one or more mass storage devices 324 are illustrated separately, however, the one or more memory devices 302 and the one or more mass storage devices 324 can be regions within the same memory module. The computing device 300 can include one or more additional processors, memory devices, network interfaces, which may be provided separately or on the same chip or board. The one or more memory devices 302 and the one or more mass storage devices 324 can include one or more computer-readable media, including, but not limited to, non-transitory computer-readable media, RAM, ROM, hard drives, flash drives, and/or other memory devices.

The one or more memory devices 302 can store sets of instructions for applications including an operating system that can be associated with various software applications or data. For example, the one or more memory devices 302 can store sets of instructions for applications that can generate output including one or more recommendations. The one or more memory devices 302 can be used to operate various applications including a mobile operating system developed specifically for mobile devices. As such, the one or more memory devices 302 can store instructions that allow the software applications to access data including data associated with the generation of one or more recommendations associated with health data. In other embodiments, the one or more memory devices 302 can be used to operate or execute a general-purpose operating system that operates on both mobile and stationary devices, including for example, smartphones, laptop computing devices, tablet computing devices, and/or desktop computers.

The software applications that can be operated or executed by the computing device 300 can include applications associated with the system 100 shown in FIG. 1A. Further, the software applications that can be operated and/or executed by the computing device 300 can include native applications and/or web-based applications.

The location device 332 can include one or more devices or circuitry for determining the position of the computing device 300. For example, the location device 332 can determine an actual and/or relative position of the computing device 300 by using a satellite navigation positioning system (e.g., a GPS system, a Galileo positioning system, the GLObal Navigation satellite system (GLONASS), and/or the BeiDou Satellite Navigation and Positioning system), an inertial navigation system, a dead reckoning system, based on IP address, by using triangulation and/or proximity to cellular towers and/or Wi-Fi hotspots. The location device 332 can be used in the generation of user metrics including the steps taken by a user and/or a distance travelled by a user.

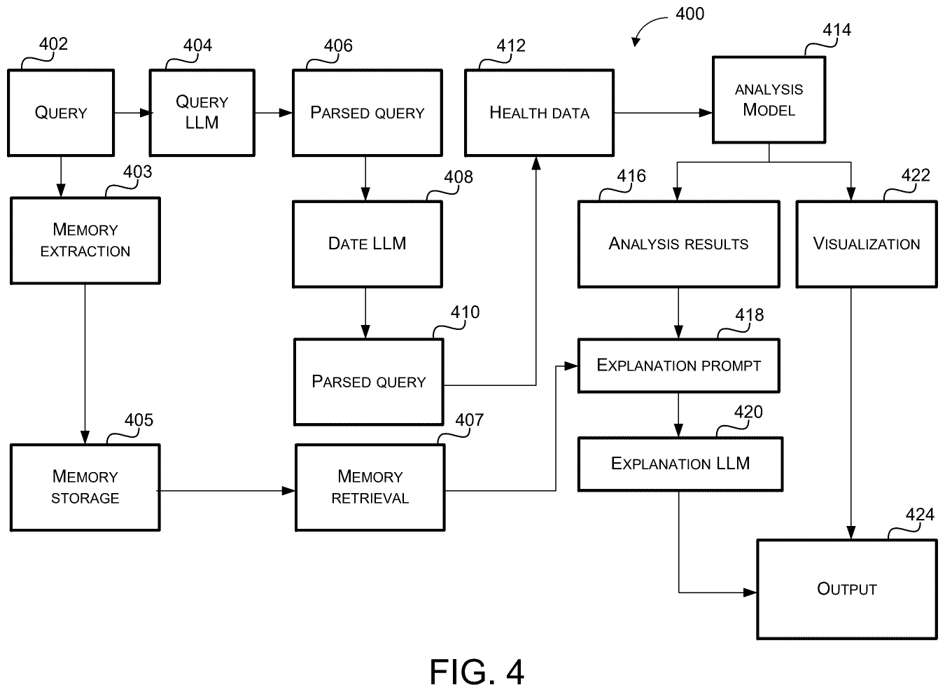

FIG. 4 depicts a block diagram of examples of operations performed by machine-learned models according to example embodiments of the present disclosure. A computing system 400 can perform one or more operations and/or one or more actions described with respect to FIG. 4. The computing system 400 can implement one or more machine-learned models that are configured and/or trained to perform the one or more operations and/or one or more actions described with respect to FIG. 4. Further, the computing system 400 can include one or more features and/or capabilities of the computing device 102, the server computing system 130, the training computing system 150, the computing device 300, and/or the computing device 500.

In some implementations, based on receiving query 402 (e.g., one or more queries associated with health data of a user), a query LLM 404 (e.g., a machine-learned model configured and/or trained to process (e.g., parse) the query 402 and/or determine one or more topics, one or more key metrics, and/or one or more analytical techniques based on input comprising the query 402) can generate parsed query 406.

In some embodiments, the memory extraction operations 403 can be performed on the query 402. The memory extraction operations 403 can extract one or more portions of the query 402 and store the one or more portions of the query 402 in one or more storage devices (e.g., SSDs and/or HDDs) by performing the memory storage operations 405. For example, the memory extraction operations 403 can extract one or more conversations associated with the health of a user and store the one or more conversations in memory by performing memory storage operations 405. As part of memory storage operations 405, the computing system 400 can index and/or organize the one or more conversations. For example, the computing system 400 can organize the one or more conversations based on the date of a conversation, the user associated with a conversation, or the class of conversation (e.g., fitness, sleep, or nutrition). In some embodiments, the computing system 400 can generate a plurality of embeddings based on the one or more conversations associated with the query 402. The plurality of embeddings can be arranged such that semantically similar content is closer together.

Further, the computing system 400 can perform memory retrieval operations 407 in which one or more queries and/or one or more conversations (e.g., previously stored queries and/or conversations) that were stored in the memory storage operations 405 are retrieved from the storage devices. Further, the computing system 400 can determine the one or more conversations that are relevant to the query 402 and add the one or more conversations that are relevant to the explanation prompt 418 before the explanation prompt is inputted into the explanation LLM model 420. For example, if the query 402 is associated with questions about a user's sleep patterns in the past two weeks, the computing system 400 can retrieve relevant conversations about the user's sleep patterns that were stored in the past two weeks and use the conversations as part of the input to the explanation LLM model 420.

The parsed query 406 can comprise one or more topics, one or more key metrics, and/or one or more analytical techniques. Further, the parsed query 406 can be used as an input to date LLM 408 (e.g., a machine-learned model that is configured and/or trained to determine one or more dates and/or date ranges based on the parsed query 406) which can generate parsed query 410. The parsed query 410 can comprise the one or more date ranges, the one or more topics, one or more key metrics, and/or one or more analytical techniques. Further, the parsed query 410, the health data 412, and/or one or more conversations and/or one or more queries retrieved as part of the memory retrieval operations 407 can be used as an input to the analysis model 414.

In some embodiments, the analysis model 414 can comprise one or more algorithms that use the one or more date ranges, the one or more topics, one or more key metrics, and/or one or more analytical techniques in the parsed query 410 to perform an analysis (e.g., statistical analysis) of the health data and generate the analysis results 416. In some embodiments, the analysis model 414 can comprise one or more machine-learned models that are configured and/or trained to generate the analysis results 416 based on input comprising the parsed query 410 and/or the health data 412. The analysis results 416 can comprise a statistical analysis of the health data 412 that is based on the query 402. The analysis results 416 can be included in an explanation prompt 418 that can be used as an input to the explanation LLM model 420. The explanation LLM model 420 (e.g., a machine-learned model that is configured and/or trained to generate one or more natural language explanations of the analysis results 416) can generate output 424 which can include an analysis and one or more explanations of the analysis results 416. Further, the analysis model 414 can be configured to generate the visualization 422 which can include one or more visualizations (e.g., graphs and/or charts) associated with the analysis results 416. The visualization 422 can be included in the output 424.

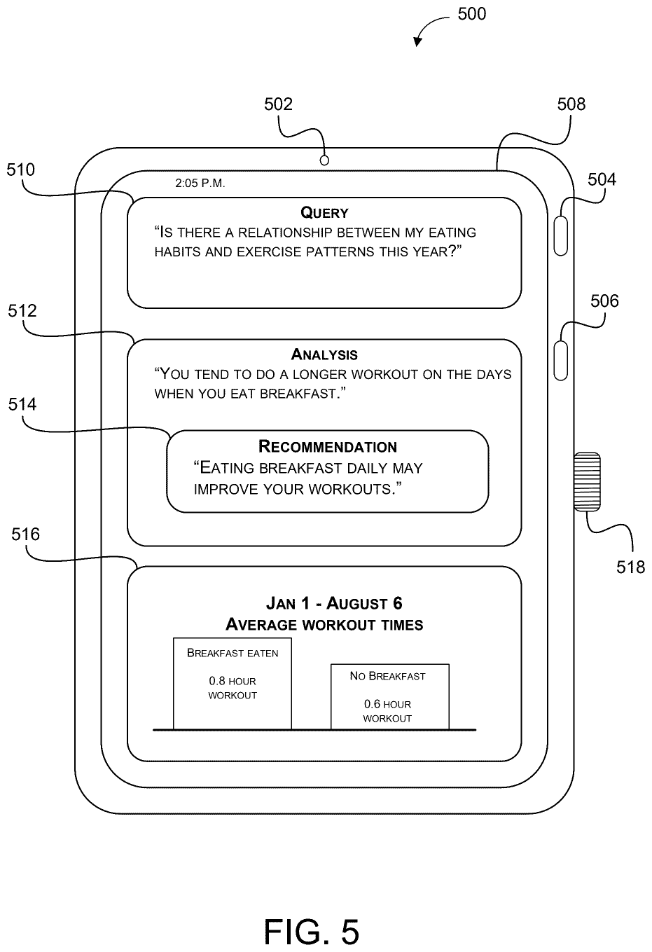

FIG. 5 depicts an example of a computing device and user interface for generating health analyses and visualizations according to example embodiments of the present disclosure. A computing device 500 can include one or more features and/or capabilities of the computing device 102, the server computing system 130, the training computing system 150, and/or the computing device 300. Furthermore, the computing device 500 can perform one or more actions and/or operations that can be performed by the computing device 102, the server computing system 130, the training computing system 150, and/or the computing device 300.

As shown in FIG. 5, the computing device 500 includes an imaging component 502, an audio input component 504, an audio output component 506, a display component 508, a query 510, an analysis 512, a recommendation 514, a visualization 516, and a tactile input component 518.

The computing device 500 can be configured to perform one or more operations comprising sending, receiving, processing, and/or generating data comprising health data and/or other data received by the computing device 500 (e.g., data associated with one or more queries). In some embodiments, the computing device 500 can comprise a mobile computing device (e.g., a wearable computing device and/or a smartphone) that can be configured to process data locally and/or receive data from a remote source (e.g., a remote computing device that is configured to store and/or processes data that can comprise health data). The data (e.g., health data) and/or query 510 received by the computing device 500 can be used to generate output comprising an analysis (e.g., the analysis 512) that can comprise a recommendation (e.g., the recommendation 514). Further, the computing device 500 can generate a visualization (e.g., visualization 516) that can comprise one or more images (e.g., charts and/or infographics) that are associated with the analysis 512 and/or recommendation 514.

Further, the computing device 500 can implement an interface (e.g., a graphical user interface) that is configured to receive one or more inputs (e.g., touch inputs via the display component 508 which can be configured to detect touch inputs and/or vocal inputs via the audio input component 504 which can comprise a microphone) from a user and perform operations which can comprise generating the analysis 512, the recommendation 514, and/or the visualization 516. The one or more inputs can be used to generate the query 510. For example, one or more touch inputs can be used to generate the query 510 via an on-screen keyboard that is generated on the display component 508. In some embodiments, the tactile input component 518 (e.g., a watch crown) can be used to navigate the interface and/or select one or more interface elements. Further, in some embodiments the tactile input component 518 can comprise a fingerprint sensor that may be used to determine whether the user of the computing device 500 is authorized to access the computing device 500 and/or generate the query 510.

In some embodiments, the analysis 512 and/or the recommendation 514 can be generated in the form of audio (e.g., a synthetic voice that reads the analysis 512 and/or recommendation 514) that is outputted via the audio output component 506 (e.g., a loudspeaker) of the computing device 500.

In this example, the computing device 500 has received the query 510, which is displayed on the display component 508. The query 510 indicates “IS THERE A RELATIONSHIP BETWEEN MY EATING HABITS AND EXERCISE PATTERNS THIS YEAR?” The computing device 500 can use the query 510 and/or health data associated with the query 510 as an input to one or more machine-learned models that are implemented on the computing device 500 and/or that are implemented on a remote computing device that is configured to send data to and/or receive data from the computing device 500. The one or more machine-learned models can be configured to process (e.g., parse) the query 510, access health data (e.g., health data associated with the user that sent the query 510), and generate output comprising one or more topics, one or more key metrics, and/or one or more analytical techniques that can be used to determine the analysis 512, the recommendation 514, and/or the visualization 516.

Based on processing the query 510, the one or more machine-learned models implemented on the computing device 500 can determine that the query is associated with one or more topics comprising relationships between metrics associated with eating habits and metrics associated with exercise patterns of the user within a defined time interval (e.g., “THIS YEAR”). Further, the one or more machine-learned models implemented by the computing device 500 can determine one or more key metrics from the health data that are associated with the one or more topics. For example, the one or more key metrics can comprise the dates and/or times of day at which various meals were consumed; the dates and/or times at which a user exercised (e.g., outdoor running and running on a treadmill); and/or the durations of exercise sessions performed by a user. Based on the one or more topics and the one or more key metrics the one or more machine-learned models implemented on the computing device 500 can determine one or more analytical techniques (e.g., regression analysis) that can be used to determine one or more analytical results (e.g., analytical results that indicate a relationship between eating habits and exercise sessions).

The one or more analytical results can be used as an input to one or more machine-learned models that are configured to generate an analysis comprising one or more explanations and the recommendation 514 and implemented on the computing device 500. The analysis 512 can be displayed on the display component 508 and can comprise a natural language explanation of the one or more analytical results. In this example, the analysis 512 indicates “YOU TEND TO DO A LONGER WORKOUT ON THE DAYS WHEN YOU EAT BREAKFAST.” Further, the analysis 512 can comprise the recommendation 514 which can comprise an actionable recommendation. In this example, the recommendation 514 indicates “EATING BREAKFAST DAILY MAY IMPROVE YOUR WORKOUTS.”

The computing device 500 can generate the visualization 516 which can graphically present the information indicated in the analysis 512. The visualization 516 can be displayed on the display component 508 and can comprise one or more images (e.g., charts, graphs, and/or infographics) based on the analysis 512. In this example, the visualization 516 comprises a time interval “JAN 1-AUGUST 6” indicating the dates (e.g., from January 1^{st} at the beginning of the year to August 6^{th} which is the date the query 510 was sent) associated with the key metrics that were used in the analysis 512 are used. Further, the visualization 516 comprises a title “AVERAGE WORKOUT TIMES” that indicates the type of information that is presented in the visualization 516. The visualization 516 also comprises a bar chart with a first bar indicating “BREAKFAST EATEN 0.8 HOUR WORKOUT” that indicates the average workout time on days when breakfast was eaten by the user, and a second bar indicating “NO BREAKFAST 0.6 HOUR WORKOUT” which indicates the average workout time on days when the user did not cat breakfast.

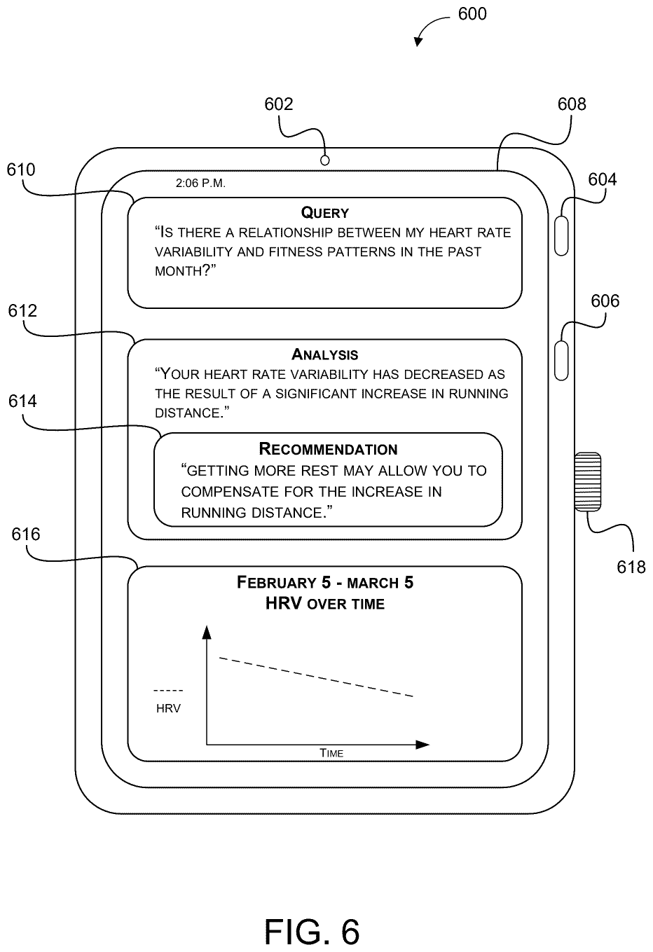

FIG. 6 depicts an example of a computing device and user interface for generating health analyses and visualizations according to example embodiments of the present disclosure. A computing device 600 can include one or more features and/or capabilities of the computing device 102, the server computing system 130, the training computing system 150, the computing device 300, and/or the computing device 500. Furthermore, the computing device 600 can perform one or more actions and/or operations that can be performed by the computing device 102, the server computing system 130, the training computing system 150, the computing device 300, and/or the computing device 500.

As shown in FIG. 6, the computing device 600 includes an imaging component 602, an audio input component 604, an audio output component 606, a display component 608, a query 610, an analysis 612, a recommendation 614, a visualization 616, and a tactile input component 618. The computing device 600 can be configured to perform one or more operations that comprise generating output comprising an analysis (e.g., the analysis 612) that can comprise a recommendation (e.g., the recommendation 614). Further, the computing device 600 can generate a visualization (e.g., visualization 616) that can comprise one or more images (e.g., charts and/or infographics) that are associated with the analysis 612 and/or recommendation 614.

In this example, the computing device 600 has received the query 610, which is displayed on the display component 608. The query 610 indicates “IS THERE A RELATIONSHIP BETWEEN MY HEART RATE VARIABILITY AND FITNESS PATTERNS IN THE PAST MONTH?” The computing device 600 can use the query 610 and/or health data associated with the query 610 as an input to one or more machine-learned models that are implemented on the computing device 600 and/or that are implemented on a remote computing device that is configured to send data to and/or receive data from the computing device 600. The one or more machine-learned models can be configured to process the query 610, access health data associated with the user that sent the query 610, and generate output comprising one or more topics, one or more key metrics, and/or one or more analytical techniques that can be used to determine the analysis 612, the recommendation 614, and/or the visualization 616. Based on processing the query 610, the one or more machine-learned models implemented on the computing device 600 can determine that the query is associated with one or more topics comprising relationships between metrics associated with heart rate variability and metrics associated with fitness patterns (e.g., the types of exercised performed by a user, step count of a user, and/or the daily duration of fitness activity) of the user over a specific time interval (e.g., “THE PAST MONTH”). Further, the one or more machine-learned models implemented by the computing device 600 can determine one or more key metrics from the health data that are associated with the one or more topics. For example, the one or more key metrics can comprise the daily average heart rate variability of the user over the one-month time interval (e.g., the past month) and/or the daily exercise duration for various exercises (e.g., running) performed by a user over the one-month time interval. Based on the one or more topics and the one or more key metrics the one or more machine-learned models implemented on the computing device 600 can determine one or more analytical techniques (e.g., regression analysis) that can be used to determine one or more analytical results (e.g., analytical results that indicate a relationship between heart rate variability and fitness patterns).

The one or more analytical results can be used as an input to one or more machine-learned models that are configured to generate an analysis comprising one or more explanations and the recommendation 614 and implemented on the computing device 600. The analysis 612 can be displayed on the display component 608 and can comprise a natural language explanation of the one or more analytical results. In this example, the analysis 612 indicates “YOUR HEART RATE VARIABILITY HAS DECREASED AS THE RESULT OF A SIGNIFICANT INCREASE IN RUNNING DISTANCE.” Further, the analysis 612 can comprise the recommendation 614 which can comprise an actionable recommendation that can assist a user in addressing the trend indicated in the analysis 612. In this example, the recommendation 614 indicates “GETTING MORE REST MAY ALLOW YOU TO COMPENSATE FOR THE INCREASE IN RUNNING DISTANCE.”

The computing device 600 can generate the visualization 616 which can graphically present the information indicated in the analysis 612. The visualization 616 can be displayed on the display component 608 and can comprise one or more images (e.g., charts, graphs, and/or infographics) based on the analysis 612. In this example, the visualization 616 comprises a time interval “FEBRUARY 5-MARCH 5” indicating the dates (e.g., from February 5^{th} (one month prior to the date the query 610 was sent) to March 5^{th} which is the date the query 610 was sent) associated with the key metrics that were used in the analysis 612 are used. Further, the visualization 616 comprises a title “HRV OVER TIME” that indicates the type of information that is presented in the visualization 616. The visualization 616 also comprises a line chart with a horizontal axis indicating “TIME” and a vertical axis indicating “HRV.” The line chart indicates that the heart rate variability has declined over the one-month time interval.

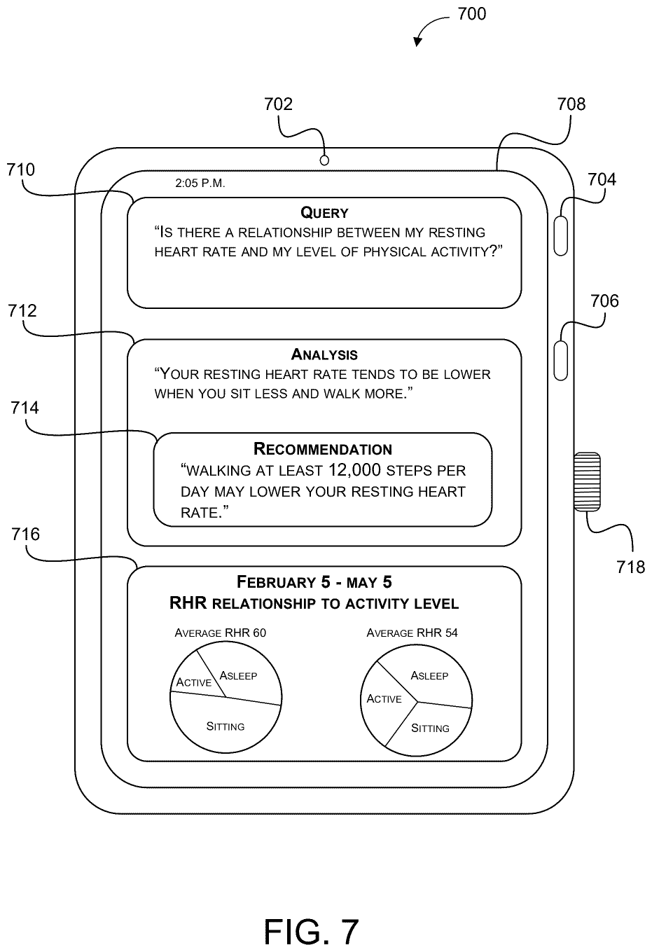

FIG. 7 depicts an example of a computing device and user interface for generating health analyses and visualizations according to example embodiments of the present disclosure. A computing device 700 can include one or more features and/or capabilities of the computing device 102, the server computing system 130, the training computing system 150, the computing device 300, and/or the computing device 500. Furthermore, the computing device 700 can perform one or more actions and/or operations that can be performed by the computing device 102, the server computing system 130, the training computing system 150, the computing device 300, and/or the computing device 500.

As shown in FIG. 7, the computing device 700 includes an imaging component 702, an audio input component 704, an audio output component 706, a display component 708, a query 710, an analysis 712, a recommendation 714, a visualization 716, and a tactile input component 718. The computing device 700 can be configured to perform one or more operations comprising generating an analysis (e.g., the analysis 712) that can comprise a recommendation (e.g., the recommendation 714). Further, the computing device 700 can generate a visualization (e.g., visualization 716) that can comprise one or more images (e.g., graphs and/or infographics) that are associated with the analysis 712 and/or recommendation 714.

In this example, the computing device 700 has received the query 710, which is displayed on the display component 708. The query 710 indicates “IS THERE A RELATIONSHIP BETWEEN MY RESTING HEART RATE AND MY LEVEL OF PHYSICAL ACTIVITY?” The computing device 700 can use the query 710 and/or health data associated with the query 710 as an input to one or more machine-learned models that are implemented on the computing device 700 and/or that are implemented on a remote computing device that is configured to send data to and/or receive data from the computing device 700. The one or more machine-learned models can be configured to process the query 710, access health data associated with the user that sent the query 710, and generate output comprising one or more topics, one or more key metrics, and/or one or more analytical techniques that can be used to determine the analysis 712, the recommendation 714, and/or the visualization 716. Based on processing the query 710, the one or more machine-learned models implemented on the computing device 700 can determine that the query is associated with one or more topics comprising relationships between metrics associated with resting heart rates and metrics associated with physical activity of the user. A time interval is not indicated in the query 710 and the computing device 700 may use health data from a default time interval (e.g., the past year, the past month, or the past three months) to generate the analysis 712. In this example, the default time interval can be a three-month time interval that ends on the day the query 710 was sent. In some embodiments, the default time interval may be defined by a user (e.g., a user determines that the default time interval will be three months). Further, the one or more machine-learned models implemented by the computing device 700 can determine one or more key metrics from the health data associated with the one or more topics. For example, the one or more key metrics can comprise the dates and/or times of day at which various meals were consumed; the dates and/or times at which a user exercised (e.g., outdoor running and running on a treadmill); and/or the durations of exercise sessions performed by a user. Based on the one or more topics and the one or more key metrics the one or more machine-learned models implemented on the computing device 700 can determine one or more analytical techniques (e.g., regression analysis) that can be used to determine one or more analytical results (e.g., analytical results that indicate a relationship between eating habits and exercise sessions).

The one or more analytical results can be used as an input to one or more machine-learned models that are configured to generate an analysis comprising one or more explanations and the recommendation 714 and implemented on the computing device 700. The analysis 712 can be displayed on the display component 708 and can comprise a natural language explanation of the one or more analytical results. In this example, the analysis 712 indicates “YOUR RESTING HEART RATE TENDS TO BE LOWER WHEN YOU SIT LESS AND WALK MORE.” Further, the analysis 712 can comprise the recommendation 714 which can comprise an actionable recommendation. In this example, the recommendation 714 indicates “WALKING AT LEAST 12,000 STEPS PER DAY MAY LOWER YOUR RESTING HEART RATE.”

The computing device 700 can generate the visualization 716 which can graphically present the information indicated in the analysis 712. The visualization 716 can be displayed on the display component 708 and can comprise one or more images (e.g., charts, graphs, and/or infographics) based on the analysis 712. In this example, the visualization 716 comprises a default time interval “FEB 5-MAY 5” indicating the dates (e.g., from February 5^{th} three months prior to receiving the query 710 to May 5^{th} which is the date the query 710 was sent) associated with the key metrics that were used in the analysis 712 are used. Further, the visualization 716 comprises a title “RHR RELATIONSHIP TO ACTIVITY LEVEL” that indicates the type of information that is presented in the visualization 716.

The visualization 716 can also comprise two pie charts. The first pie chart (the pic chart on the left side of the visualization 716) can indicate “AVERAGE RHR 60” which is the average resting heart rate of the user over the default time interval. Further, the first pie chart can show the distribution of the average amount of time per day that a user is active, asleep, or sitting when the resting heart rate is 60 beats per minute. In the first pie chart, the user is active for approximately one sixth of the day (e.g., approximately 4 hours per day), asleep for approximately one third of the day (e.g., approximately 8 hours per day), and sits for approximately half of the day (e.g., approximately 12 hours per day).

The second pie chart (the pie chart on the right side of the visualization 716) can indicate “AVERAGE RHR 54” which is the average resting heart rate of the user over the default time interval. Further, the second pie chart can show the distribution of the average amount of time per day that a user is active, asleep, or sitting when the resting heart rate is 54 beats per minute. In the second pie chart, the user is active for approximately one third of the day (e.g., approximately 8 hours per day), asleep for approximately one third of the day (e.g., approximately 8 hours per day), and sits for approximately one third of the day (e.g., approximately 8 hours per day). The visualization 716 can show that a user's resting heart rate is lower when the user is more active.

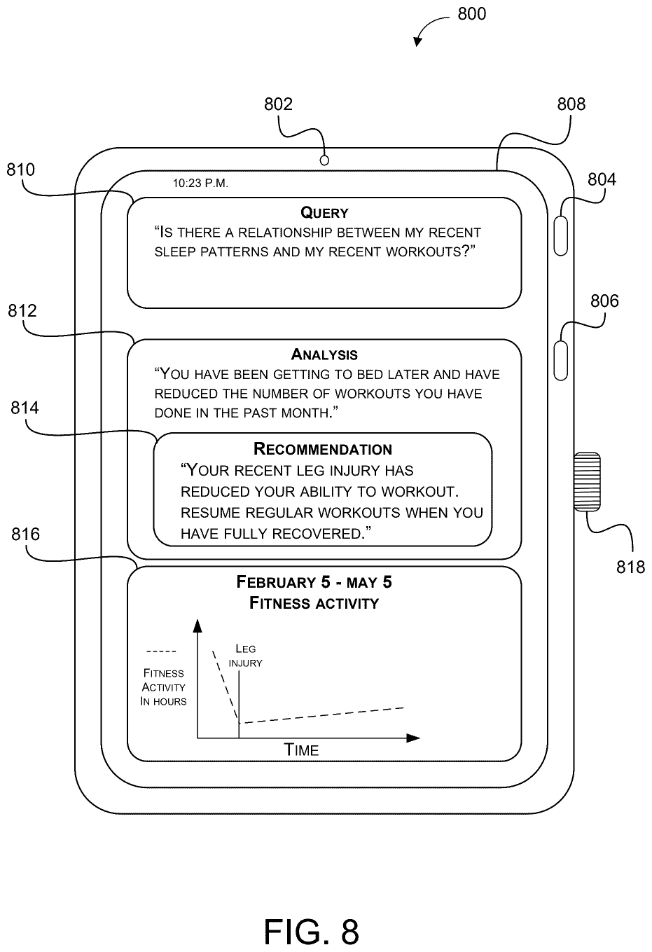

FIG. 8 depicts an example of a computing device and user interface for generating health analyses and visualizations according to example embodiments of the present disclosure. A computing device 800 can include one or more features and/or capabilities of the computing device 102, the server computing system 130, the training computing system 150, and/or the computing device 300. Furthermore, the computing device 800 can perform one or more actions and/or operations that can be performed by the computing device 102, the server computing system 130, the training computing system 150, the computing device 300, and/or the computing device 500.

As shown in FIG. 8, the computing device 800 includes an imaging component 802, an audio input component 804, an audio output component 806, a display component 808, a query 810, an analysis 812, a recommendation 814, a visualization 816, and a tactile input component 818. The computing device 800 can be configured to perform one or more operations comprising generating an analysis (e.g., the analysis 812) that can comprise a recommendation (e.g., the recommendation 814). Further, the computing device 800 can generate a visualization (e.g., visualization 816) that can comprise one or more images (e.g., graphs and/or infographics) that are associated with the analysis 812 and/or recommendation 814.

In this example, the computing device 800 has received the query 810, which is displayed on the display component 808. The query 810 indicates “IS THERE A RELATIONSHIP BETWEEN MY RECENT SLEEP PATTERNS AND MY RECENT WORKOUTS?” The computing device 800 can use the query 810 and/or health data associated with the query 810 as an input to one or more machine-learned models that are implemented on the computing device 800 and/or that are implemented on a remote computing device that is configured to send data to and/or receive data from the computing device 800. The one or more machine-learned models can be configured to process the query 810, access health data associated with the user that sent the query 810, and generate output comprising one or more topics, one or more key metrics, and/or one or more analytical techniques that can be used to determine the analysis 812, the recommendation 814, and/or the visualization 816. Based on processing the query 810, the one or more machine-learned models implemented on the computing device 800 can determine that the query is associated with one or more topics comprising relationships between metrics associated with sleep patterns and metrics associated with physical activity of the user. The query 810 indicates the word “RECENT” and the computing device 800 can determine that recent is within the past month and may use health data from the past month to generate the analysis 812. Further, the one or more machine-learned models implemented by the computing device 800 can determine one or more key metrics from the health data associated with the one or more topics. For example, the one or more key metrics can comprise the dates and/or times of day at which the user went to sleep and woke up; the dates and/or times at which a user exercised (e.g., rowing machine); and/or the durations of exercise sessions performed by a user. Based on the one or more topics and the one or more key metrics the one or more machine-learned models implemented on the computing device 800 can determine one or more analytical techniques (e.g., linear regression analysis) that can be used to determine one or more analytical results (e.g., analytical results that indicate a relationship between sleep patterns and fitness activity).

The one or more analytical results can be used as an input to one or more machine-learned models that are configured to generate an analysis comprising one or more explanations and the recommendation 814 and implemented on the computing device 800. The analysis 812 can be displayed on the display component 808 and can comprise a natural language explanation of the one or more analytical results. In this example, the analysis 812 indicates “YOU HAVE BEEN GETTING TO BED LATER AND HAVE REDUCED THE NUMBER OF WORKOUTS YOU HAVE DONE IN THE PAST MONTH.” Further, the analysis 812 can comprise the recommendation 814 which can comprise an actionable recommendation. In this example, the recommendation 814 indicates “YOUR RECENT LEG INJURY HAS REDUCED YOUR ABILITY TO WORKOUT. RESUME REGULAR WORKOUTS WHEN YOU HAVE FULLY RECOVERED.”

The computing device 800 can generate the visualization 816 which can graphically present the information indicated in the analysis 812. The visualization 816 can be displayed on the display component 808 and can comprise one or more images (e.g., charts, graphs, and/or infographics) based on the analysis 812. In this example, the visualization 816 comprises a time interval “FEBRUARY 5-MAY 5” indicating the dates (e.g., from February 5^{th} (three months prior to the date the query 810 was sent) to May 5^{th} which is the date the query 810 was sent) associated with the key metrics that were used in the analysis 812 are used. Further, the visualization 816 comprises a title “FITNESS ACTIVITY” that indicates the type of information that is presented in the visualization 816. The visualization 816 also comprises a line chart with a horizontal axis indicating “TIME” and a vertical axis indicating “FITNESS ACTIVITY IN HOURS” which indicates the number of hours that the user has engaged in fitness activity (e.g., running, rowing, and/or swimming). Further, the visualization 816 indicates the time at which the user suffered a “LEG INJURY.” The line chart indicates that the fitness activity significantly declined at the time the user's leg was injured, has slowly increased over the three-month time interval since the leg injury, but has not yet reached the level of fitness activity that the user engaged in prior to the leg injury.

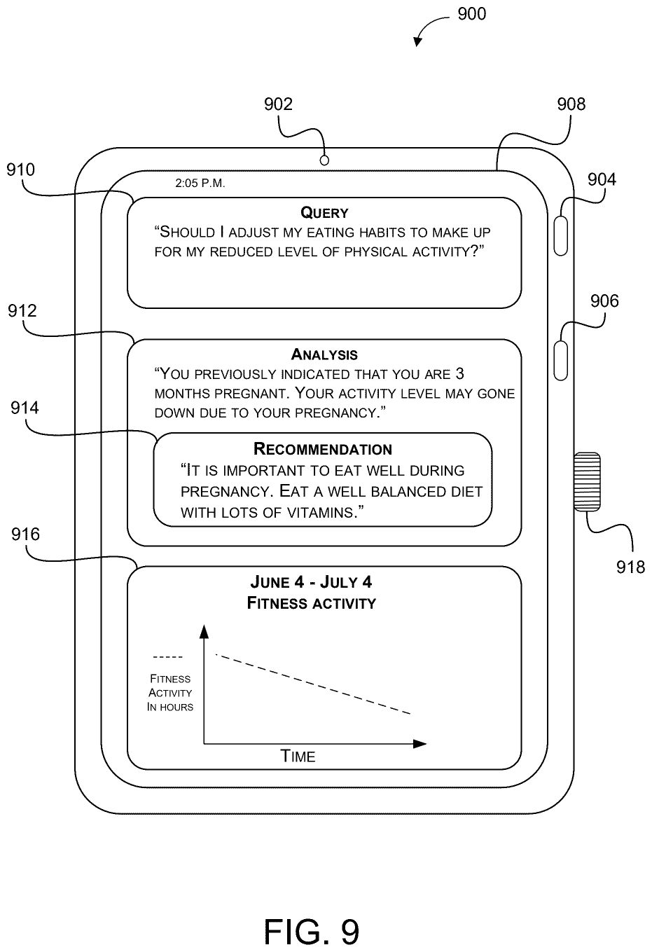

FIG. 9 depicts an example of a computing device and user interface for generating health analyses and visualizations according to example embodiments of the present disclosure. A computing device 900 can include one or more features and/or capabilities of the computing device 102, the server computing system 130, the training computing system 150, and/or the computing device 300. Furthermore, the computing device 900 can perform one or more actions and/or operations that can be performed by the computing device 102, the server computing system 130, the training computing system 150, the computing device 300, and/or the computing device 500.

As shown in FIG. 9, the computing device 900 includes an imaging component 902, an audio input component 904, an audio output component 906, a display component 908, a query 910, an analysis 912, a recommendation 914, a visualization 916, and a tactile input component 918. The computing device 900 can be configured to perform one or more operations comprising generating an analysis (e.g., the analysis 912) that can comprise a recommendation (e.g., the recommendation 914). Further, the computing device 900 can generate a visualization (e.g., visualization 916) that can comprise one or more images (e.g., graphs and/or infographics) that are associated with the analysis 912 and/or recommendation 914.

In this example, the computing device 900 has received the query 910, which is displayed on the display component 908. The query 910 indicates “SHOULD I ADJUST MY EATING HABITS TO MAKE UP FOR MY REDUCED LEVEL OF PHYSICAL ACTIVITY?” The computing device 900 can use the query 910 and/or health data associated with the query 910 as an input to one or more machine-learned models that are implemented on the computing device 900 and/or that are implemented on a remote computing device that is configured to send data to and/or receive data from the computing device 900. The one or more machine-learned models can be configured to process the query 910, access health data associated with the user that sent the query 910, access previous conversations with the user (e.g., a conversation in which the user indicated that the user is pregnant and a length of time the user has been pregnant), and generate output comprising one or more topics, one or more key metrics, and/or one or more analytical techniques that can be used to determine the analysis 912, the recommendation 914, and/or the visualization 916. Based on processing the query 910, the one or more machine-learned models implemented on the computing device 900 can determine that the query is associated with one or more topics comprising relationships between metrics associated with eating habits (e.g., nutritional habits and the times that meals are consumed) and metrics associated with physical activity of the user. A time interval is not indicated in the query 910 and the computing device 900 may use health data from a default time interval (e.g., the past month) to generate the analysis 912. In this example, the default time interval can be a one-month time interval that ends on the day the query 910 was sent. Further, the one or more machine-learned models implemented by the computing device 900 can determine one or more key metrics from the health data associated with the one or more topics. For example, the one or more key metrics can comprise the dates and/or times of day at which various meals were consumed; the dates and/or times at which a user exercised (e.g., cross-country skiing and/or cycling); and/or the durations of exercise sessions performed by a user. Based on the one or more topics and the one or more key metrics the one or more machine-learned models implemented on the computing device 900 can determine one or more analytical techniques (e.g., regression analysis) that can be used to determine one or more analytical results (e.g., analytical results that indicate a relationship between eating habits and physical activity).

The one or more analytical results can be used as an input to one or more machine-learned models that are configured to generate an analysis comprising one or more explanations and the recommendation 914 and implemented on the computing device 900. The analysis 912 can be displayed on the display component 908 and can comprise a natural language explanation of the one or more analytical results. In this example, the analysis 912 indicates “YOU PREVIOUSLY INDICATED THAT YOU ARE 3 MONTHS PREGNANT. YOUR ACTIVITY LEVEL MAY GONE DOWN DUE TO YOUR PREGNANCY.” Further, the analysis 912 can comprise the recommendation 914 which can comprise an actionable recommendation. In this example, the recommendation 914 indicates “IT IS IMPORTANT TO EAT WELL DURING PREGNANCY. EAT A WELL BALANCED DIET WITH LOTS OF VITAMINS.”

The computing device 900 can generate the visualization 916 which can graphically present the information indicated in the analysis 912. The visualization 916 can be displayed on the display component 908 and can comprise one or more images (e.g., charts, graphs, and/or infographics) based on the analysis 912. In this example, the visualization 916 comprises a time interval “JUNE 4-JULY 4” indicating the dates (e.g., from June 4^{th} (one month prior to the date the query 910 was sent) to July 4^{th} which is the date the query 910 was sent) associated with the key metrics that were used in the analysis 912 are used. Further, the visualization 916 comprises a title “FITNESS ACTIVITY” that indicates the type of information that is presented in the visualization 916. The visualization 916 also comprises a line chart with a horizontal axis indicating “TIME” and a vertical axis indicating “FITNESS ACTIVITY IN HOURS” which indicates the number of hours that the user has engaged in fitness activity (e.g., walking, stair climbing, and/or yoga). Further, the visualization 916 indicates the time at which the user suffered a “LEG INJURY.” The line chart indicates that the fitness activity has slowly declined over the pregnancy of the user.

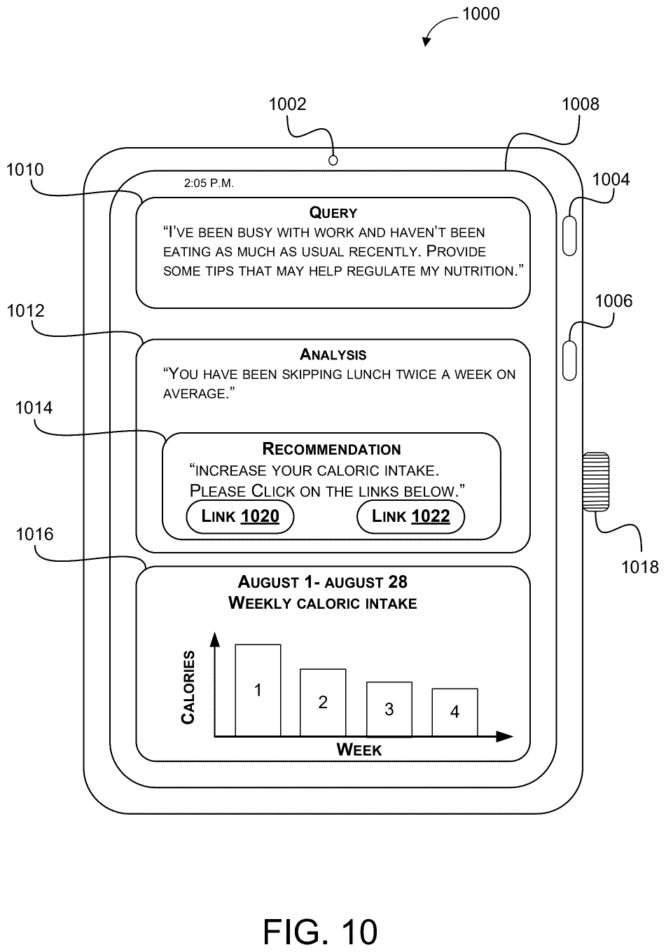

FIG. 10 depicts an example of a computing device and user interface for generating health analyses and visualizations according to example embodiments of the present disclosure. A computing device 1000 can include one or more features and/or capabilities of the computing device 102, the server computing system 130, the training computing system 150, and/or the computing device 300. Furthermore, the computing device 1000 can perform one or more actions and/or operations that can be performed by the computing device 102, the server computing system 130, the training computing system 150, the computing device 300, and/or the computing device 500.

As shown in FIG. 10, the computing device 1000 includes an imaging component 1002, an audio input component 1004, an audio output component 1006, a display component 1008, a query 1010, an analysis 1012, a recommendation 1014, a visualization 1016, and a tactile input component 1018. The computing device 1000 can be configured to perform one or more operations comprising generating an analysis (e.g., the analysis 1012) that can comprise a recommendation (e.g., the recommendation 1014). Further, the computing device 1000 can generate a visualization (e.g., visualization 1016) that can comprise one or more images (e.g., graphs and/or infographics) that are associated with the analysis 1012 and/or recommendation 1014.

In this example, the computing device 1000 has received the query 1010, which is displayed on the display component 1008. The query 1010 indicates “I'VE BEEN BUSY WITH WORK AND HAVEN'T BEEN EATING AS MUCH AS USUAL RECENTLY. PROVIDE SOME TIPS THAT MAY HELP REGULATE MY NUTRITION.” The computing device 1000 can use the query 1010 and/or health data associated with the query 1010 as an input to one or more machine-learned models that are implemented on the computing device 1000 and/or that are implemented on a remote computing device that is configured to send data to and/or receive data from the computing device 1000. The one or more machine-learned models can be configured to process the query 1010, access health data associated with the user that sent the query 1010, and generate output comprising one or more topics, one or more key metrics, and/or one or more analytical techniques that can be used to determine the analysis 1012, the recommendation 1014, and/or the visualization 1016. Based on processing the query 1010, the one or more machine-learned models implemented on the computing device 1000 can determine that the query is associated with one or more topics comprising relationships between metrics associated with eating habits of the user. A specific time interval is not indicated in the query 1010 and the computing device 1000 may parse the query 1010 and determine that the term “RECENTLY” can include a four week time interval. Further, the one or more machine-learned models implemented by the computing device 1000 can determine one or more key metrics from the health data associated with the one or more topics. For example, the one or more key metrics can comprise the dates and/or times of day at which various meals were consumed; the dates and/or times at which a user exercised (e.g., outdoor running and running on a treadmill); and/or the durations of sleep by a user. Based on the one or more topics and the one or more key metrics the one or more machine-learned models implemented on the computing device 1000 can determine one or more analytical techniques (e.g., regression analysis) that can be used to determine one or more analytical results (e.g., analytical results that indicate anomalies in the user's eating habits).

The one or more analytical results can be used as an input to one or more machine-learned models that are configured to generate an analysis comprising one or more explanations and the recommendation 1014 and implemented on the computing device 1000. The analysis 1012 can be displayed on the display component 1008 and can comprise a natural language explanation of the one or more analytical results. In this example, the analysis 1012 indicates “YOU HAVE BEEN SKIPPING LUNCH TWICE A WEEK ON AVERAGE.” Further, the analysis 1012 can comprise the recommendation 1014 which can comprise an actionable recommendation. In this example, the recommendation 1014 indicates “INCREASE YOUR CALORIC INTAKE. PLEASE CLICK ON THE LINKS BELOW.” The computing device 1000 can generate links to external sources, documents, and/or citations that can be used to support the recommendation 1014 and/or provide additional information that may be relevant to the query 1010. In this example, the link 1020 can comprise a link to a website that includes information about good eating habits and/or tips on finding time to cat during busy work periods. The link 1022 can comprise a link to a document that includes recent research on nutrition and the effects of skipping meals. In some embodiments, the computing device 1000 can generate links to health websites, medical information websites, nutrition websites, fitness websites, and/or document repositories that can provide relevant research documents.

The computing device 1000 can generate the visualization 1016 which can graphically present the information indicated in the analysis 1012. The visualization 1016 can be displayed on the display component 1008 and can comprise one or more images (e.g., charts, graphs, and/or infographics) based on the analysis 1012. In this example, the visualization 1016 comprises a time interval “AUG 1-AUGUST 28” indicating the dates (e.g., from August 1^{st} to August 28^{th} which is the date the query 1010 was sent) associated with the key metrics that were used in the analysis 1012 are used. Further, the visualization 1016 comprises a title “WEEKLY ACTIVITY AND CALORIC INTAKE” that indicates the type of information that is presented in the visualization 1016 (e.g., the aggregate caloric intake in kcals per week). The visualization 1016 also comprises a bar chart with four bars “1,” “2,” “3,” and “4,” which indicate the week. Further, the height of each bar in the bar chart indicates the caloric intake of the user during the respective week. The bar chart indicates that the caloric intake of the user has gradually gone down over time.

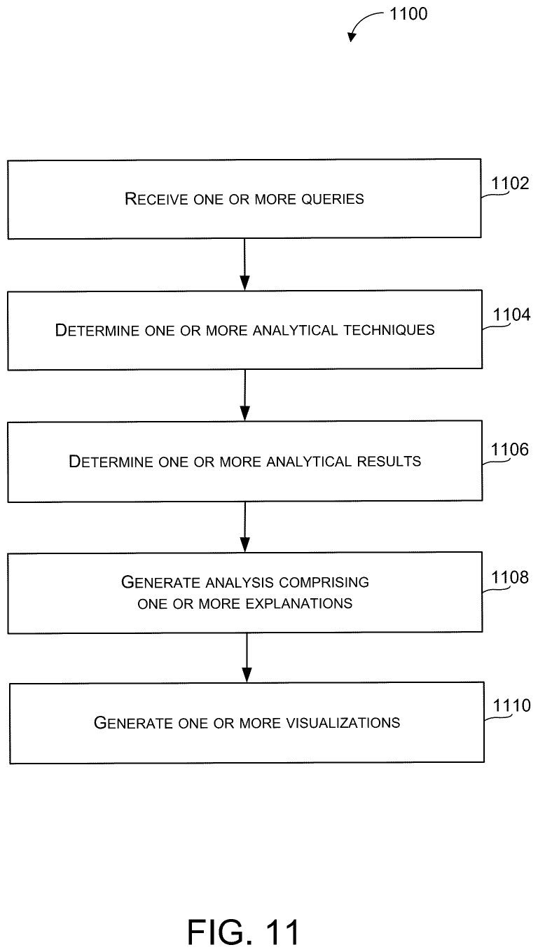

FIG. 11 depicts a flow chart diagram of an example method to generate health analyses and visualizations according to example embodiments of the present disclosure. One or more portions of the method 1100 can be executed and/or implemented on one or more computing devices or computing systems comprising, for example, the computing device 102, the server computing system 130, the training computing system 150, and/or the computing device 300, and/or the computing device 500. Further, one or more portions of the method 1100 can be executed or implemented as an algorithm on the hardware devices or systems disclosed herein. FIG. 11 depicts steps performed in a particular order for purposes of illustration and discussion. Those of ordinary skill in the art, using the disclosures provided herein, will understand that various steps of any of the methods disclosed herein can be adapted, modified, rearranged, omitted, and/or expanded without deviating from the scope of the present disclosure.

At 1102, the method 1100 can include receiving one or more queries. The one or more queries can be associated with health data. Further, the health data can comprise health information (e.g., health information associated with the user associated with the health data). For example, the computing device 102 can receive query data that comprises the one or more queries and which is inputted into a health and fitness application that is operational on the computing device 102.

At 1104, the method 1100 can include determining, based on inputting the one or more queries into one or more machine-learned models, one or more topics of the one or more queries, one or more key metrics of the health data, and/or one or more analytical techniques based on the one or more topics and the one or more key metrics. For example, the computing device 102 can perform one or more operations comprising using the health data as part of an input to one or more machine-learned models that are configured to receive the input, perform one or more operations on the input, and generate an output comprising the one or more topics of the one or more queries, the one or more key metrics of the health data, and/or the one or more analytical techniques.

The one or more machine-learned models can be configured and/or trained to process and/or parse the health data to determine one or more topics of the one or more queries, one or more key metrics of the health data, and/or one or more analytical techniques. For example, the one or more machine-learned models can comprise one or more large language models that are configured and/or trained to determine the one or more topics that are associated with one or more portions of one or more queries. Further, the one or more machine-learned models can determine one or more key metrics of the health data that are associated with the one or more topics. Further, the one or more machine-learned models can determine one or more analytical techniques that are based on the topics and the key metrics that were determined. For example, one or more queries that request information about sleep patterns may result in one or more topics associated with sleep patterns, one or more key metrics associated with sleep patterns (e.g., nightly sleep time or bedtime), and one or more analytical techniques that can be used to determine correlations between sleep patterns and other health metrics, activity metrics, and/or nutritional metrics.

At 1106, the method 1100 can include determining one or more analytical results. The one or more analytical results can be based on performing the one or more analytical techniques on at least the user data comprising the one or more key metrics. For example, the computing device 102 can use one or more statistical analysis techniques (e.g., trend analysis) on one or more key metrics (e.g., nightly sleep duration) to determine the one or more analytical results comprising an increase in a user's sleep duration over a time interval comprising the previous two months.

At 1108, the method 1100 can include generating one or more explanations of the one or more analytical results. Generating the one or more explanations can be based on inputting the one or more analytical results into the one or more machine-learned models. For example, the computing device 102 can implement one or more machine-learned models that are configured and/or trained to receive input comprising the one or more explanations, the one or more queries, and/or the health data, perform one or more operations on the input, and generate an output comprising the one or more explanations. Further, the computing device 102 can display one or more explanations (e.g., text-based explanations) on a user interface that is generated on a display device.

At 1110, the method 1100 can include generating one or more visualizations. The one or more visualizations can be based on the analysis (e.g., the analysis comprising the one or more explanations). For example, the computing device 102 can display one or more visualizations (e.g., line charts that are based on the one or more explanations) on a user interface that is generated on a display device.

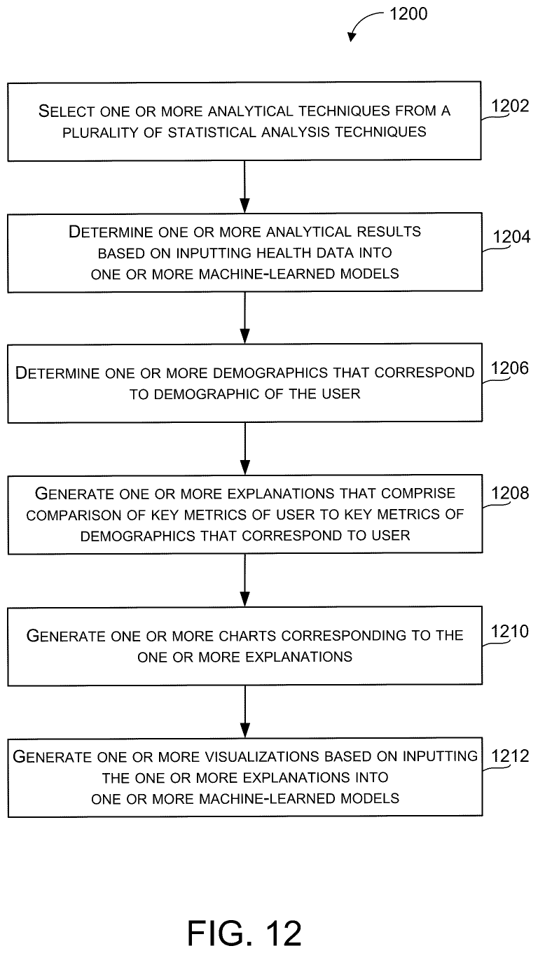

FIG. 12 depicts a flow chart diagram of an example method to generate health analyses and visualizations according to example embodiments of the present disclosure. One or more portions of the method 1200 can be executed and/or implemented on one or more computing devices or computing systems comprising, for example, the computing device 102, the server computing system 130, the training computing system 150, and/or the computing device 300, and/or the computing device 500. Further, one or more portions of the method 1200 can be executed or implemented as an algorithm on the hardware devices or systems disclosed herein. In some embodiments, one or more portions of the method 1200 can be performed as part of the method 1100 that is described with respect to FIG. 11. FIG. 12 depicts steps performed in a particular order for purposes of illustration and discussion. Those of ordinary skill in the art, using the disclosures provided herein, will understand that various steps of any of the methods disclosed herein can be adapted, modified, rearranged, omitted, and/or expanded without deviating from the scope of the present disclosure.

At 1202, the method 1200 can include selecting the one or more analytical techniques from a plurality of statistical analysis techniques. Selection of the one or more analytical techniques can be based on the one or more topics and/or the one or more key metrics. For example, the computing device 102 can implement one or more machine-learned models that are configured to determine the one or more analytical techniques that are associated with the one or more queries.

At 1204, the method 1200 can include generating and/or determining the one or more analytical results based on inputting the health data comprising the one or more key metrics into the one or more machine-learned models. For example, the computing device 102 can implement one or more machine-learned models that are configured to generate the one or more analytical results based on input comprising the one or more key metrics.

At 1206, the method 1200 can include determining one or more demographics that correspond to the health data. For example, the computing device 102 can access health data that indicates an age and/or gender of the user based on information provided with the consent of the user. The demographic data (e.g., age and/or gender) of the user can be stored in a privacy enhancing manner (the health data comprising the demographic data is encrypted) and not shared without the express consent of the user.

At 1208, the method 1200 can include generating one or more explanations that can comprise one or more comparisons of the one or more key metrics of the user to the one or more key metrics of aggregate health data that corresponds to the one or more demographics. For example, the computing device 102 can compare one or more key metrics comprising the nightly sleep duration of a twenty-year-old woman to the average nightly sleep duration of hundreds of thousands of other twenty-year-old women that is included in the aggregate health data. The one or more explanations can include an indication of whether the user sleeps more or less than the average twenty-year-old woman.

At 1210, the method 1200 can include generating one or more charts corresponding to the one or more explanations. For example, the computing device 102 can generate a line chart based on one or more explanations that indicate that a user's resting heart rate has increased in the past three months.

At 1212, the method 1200 can include generating and/or determining the one or more visualizations based on inputting the one or more explanations into the one or more machine-learned models. For example, the computing device 102 can implement one or more machine-learned models that are configured to generate the one or more visualizations based on input comprising the one or more explanations.

Further to the descriptions above, a user may be provided with controls allowing the user to make an election as to both if and/or when systems, programs, or features described herein may enable collection of user information (e.g., fitness information, physical exercise activities, and/or a user's preferences), and if the user is sent content or communications from a server. In addition, certain data may be treated in one or more ways before it is stored or used, so that certain information of a user may be removed. For example, a user's identity may be treated so that certain other information associated with the user's identity may not be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure covers such alterations, variations, and equivalents.

What is claimed is:

1. A computer-implemented method of processing health data, the computer-implemented method comprising:
receiving, by a computing system comprising one or more processors, one or more queries associated with health data comprising health information;
determining, by the computing system, based on inputting the one or more queries into one or more machine-learned models, one or more topics of the one or more queries, one or more key metrics of the health data, and one or more analytical techniques based on the one or more topics and the one or more key metrics;
determining, by the computing system, based on performing the one or more analytical techniques on at least the health data comprising the one or more key metrics, one or more analytical results;
generating, by the computing system, based on inputting the one or more analytical results into the one or more machine-learned models, an analysis comprising one or more explanations of the one or more analytical results; and
generating, by the computing system, one or more visualizations based on the analysis.

2. The computer-implemented method of claim 1, wherein the one or more explanations comprise one or more natural language explanations of the one or more analytical results.

3. The computer-implemented method of claim 1, wherein the health data comprises activity information associated with one or more activities, and wherein the activity information comprises one or more times at which the one or more activities are performed.

4. The computer-implemented method of claim 1, wherein the analysis comprises one or more recommendations based on the one or more analytical results, wherein the one or more recommendations comprise one or more natural language recommendations based on at least one of the one or more key metrics.

5. The computer-implemented method of claim 1, wherein the one or more analytical results comprise one or more statistical relationships between at least one of the one or more key metrics and at least one of the one or more key metrics based on aggregate health data.

6. The computer-implemented method of claim 1, wherein the one or more machine-learned models comprise one or more large language models (LLMs) that are configured to parse the one or more queries and identify the one or more topics, the one or more key metrics, and the one or more analytical techniques.

7. The computer-implemented method of claim 1, wherein the one or more machine-learned models are configured to determine a range of dates from which the one or more key metrics are selected.

8. The computer-implemented method of claim 1, wherein the health data comprises nutritional information associated with food consumption.

9. The computer-implemented method of claim 1, wherein the determining, by the computing system, based on inputting the one or more queries into one or more machine-learned models, one or more topics of the one or more queries, one or more key metrics of the health data, and one or more analytical techniques based on the one or more topics and the one or more key metrics comprises:
selecting, by the computing system, based on the one or more topics and the one or more key metrics, the one or more analytical techniques from a plurality of statistical analysis techniques.

10. The computer-implemented method of claim 1, wherein the determining, by the computing system, based on performing the one or more analytical techniques on at least the health data comprising the one or more key metrics, one or more analytical results comprises:
determining, by the computing system, based on inputting the health data comprising the one or more key metrics into the one or more machine-learned models, the one or more analytical results.

11. The computer-implemented method of claim 1, wherein the generating, by the computing system, based on inputting the one or more analytical results into the one or more machine-learned models, an analysis comprising one or more explanations of the one or more analytical results comprises:
determining, by the computing system, one or more demographics that correspond to the health data; and
generating, by the computing system, one or more explanations comprising one or more comparisons of the one or more key metrics that correspond to the health data to the one or more key metrics of aggregate health data that corresponds to the one or more demographics.

12. The computer-implemented method of claim 1, wherein the generating, by the computing system, one or more visualizations based on the one or more explanations comprises:
generating, by the computing system, one or more charts corresponding to the one or more explanations.

13. The computer-implemented method of claim 12, wherein the one or more charts comprise one or more area charts, one or more bar charts, one or more line charts, or one or more scatter plots.

14. The computer-implemented method of claim 1, wherein the generating, by the computing system, one or more visualizations based on the one or more explanations comprises:
generating, by the computing system, based on inputting the one or more explanations into the one or more machine-learned models, the one or more visualizations.

15. One or more tangible non-transitory computer-readable media storing computer-readable instructions that when executed by one or more processors cause the one or more processors to perform operations, the operations comprising:
receiving one or more queries associated with health data comprising health information;
determining, based on inputting the one or more queries into one or more machine-learned models, one or more topics of the one or more queries, one or more key metrics of the health data, and one or more analytical techniques based on the one or more topics and the one or more key metrics;
determining, based on performing the one or more analytical techniques on at least the health data comprising the one or more key metrics, one or more analytical results;
generating, based on inputting the one or more analytical results into the one or more machine-learned models, an analysis comprising one or more explanations of the one or more analytical results; and
generating one or more visualizations based on the analysis.

16. The one or more tangible non-transitory computer-readable media of claim 15, wherein the one or more machine-learned models comprise one or more large language models (LLMs) that are configured to parse the one or more queries and identify the one or more key metrics and one or more analytical techniques.

17. The one or more tangible non-transitory computer-readable media of claim 15, wherein the health data comprises activity information associated with one or more activities, and wherein the activity information comprises one or more times at which the one or more activities are performed.

18. A computing system comprising:
one or more processors;
one or more non-transitory computer-readable media storing instructions that when executed by the one or more processors cause the one or more processors to perform operations comprising:
receiving one or more queries associated with health data comprising health information;
determining, based on inputting the one or more queries into one or more machine-learned models, one or more topics of the one or more queries, one or more key metrics of the health data, and one or more analytical techniques based on the one or more topics and the one or more key metrics;
determining, based on performing the one or more analytical techniques on at least the health data comprising the one or more key metrics, one or more analytical results;
generating, based on inputting the one or more analytical results into the one or more machine-learned models, an analysis comprising one or more explanations of the one or more analytical results; and
generating one or more visualizations based on the analysis.

19. The computing system of claim 18, wherein the one or more machine-learned models comprise one or more large language models (LLMs) that are configured to parse the one or more queries and identify the one or more key metrics and one or more analytical techniques.

20. The computing system of claim 18, wherein the health data comprises activity information associated with one or more activities, and wherein the activity information comprises one or more times at which the one or more activities are performed.
